(19) United States
(12) Patent Application Publication
(10) Pub. No.: US 2017/0283537 A1
(43) Pub. Date: Oct. 5, 2017

(54) Stable Polymeric Nanoparticle Compositions and Methods Related Thereto

(71) Applicant: MASSACHUSETTS INSTITUTE OF TECHNOLOGY, Cambridge, MA (US)

(72) Inventors: T. Alan Hatton, Sudbury, MA (US); Lev E. Bromberg, Swampscott, MA (US); Mikhil Ranka, Cambridge, MA (US)

(21) Appl. No.: 15/510,351

(22) PCT Filed: Sep. 14, 2015
(86) PCT No.: PCT/US15/49902

§ 371 Date: Mar. 10, 2017

Related U.S. Application Data
(60) Provisional application No. 62/049,490, filed on Sep. 12, 2014.

(51) Int. Cl. C08F 220/60; G01N 33/00; G01N 33/24; C08F 220/58; C08F 292/00

(52) U.S. Cl. CPC C08F 220/60; C08F 220/58; C08F 292/00; G01N 33/241; G01N 33/0044; C08F 2220/606; C08F 2220/585

(57) ABSTRACT

Disclosed are compositions comprising composite nanoparticles and a reporter molecule for use in detecting the presence of an analyte in a substrate. The composite nanoparticles comprise a solid core and a polyampholyte covalently bonded to the solid core. Reporter molecules are releasable from the composite nanoparticles upon exposure to the analyte. Analytes may include petroleum or other hydrophobic media.

### RELATED APPLICATIONS

This application claims the benefit of priority to U.S. Provisional Patent Application Ser. No. 62/049,490, filed Sep. 12, 2014; the contents of which are hereby incorporated by reference.

### BACKGROUND

Nanoparticles are solid particles having at least one dimension of less than 1 micron. Particularly desirable nanoparticles are between 10 nm and 100 nm in size. For example, engineered nanoparticles with sizes below 100 nm may be used as reporters for hydrocarbon detection in oil-field rocks. See U.S. 2012/0142111, which is incorporated herein by reference in its entirety. In particular, amphiphilic reporter nanoparticles comprising a solid core and a polymeric shell can sequester a hydrophobic compound, such as oil or a sulfur-containing compound. The nanoparticles may be injected into the subsurface, and can then transport their hydrophobic payloads through oil-field rocks. If the rocks contain oil, the payloads are selectively released. The nanoparticles may then be recovered and analyzed for remaining payload, where release of the payload indicates the presence of oil. When used in this manner the nanoparticles are known as “nanoreporters.”

Amphiphilic nanoreporters known in the art are stabilized by steric hinderance. Steric stabilization is a process by which adsorbed/covalently-attached nonionic surfactants or polymers induce osmotic and/or entropic repulsion between particles in a suspension. The adsorbtion/attachment of nonionic surfactants or polymers on the surface of particles produces an adsorbed layer, which can be strongly solvated by the solvent when the solvent is a good solvent for the surfactant or polymer layer. However, when a solution is a poor solvent for the adsorbed polymer, the nanoparticles aggregate and form clusters, thereby leading to destabilization of the suspension. The irregular particle clusters, flocs, or aggregates formed in a poor solvent are unsuitable for use as nanoreporters. For example, at certain salt concentrations aqueous salt solutions become poor solvents for nonionic surfactants or polymers. As a result, in the presence of high salt solutions, the nanoparticles known in the art aggregate, and are no longer usable as nanoreporters for oil.

Therefore, there is a need for nanoparticles exhibiting good colloidal stability in aqueous salt solutions.

### SUMMARY

In certain embodiments, the invention relates to a composite nanoparticle, comprising, consisting essentially of, or consisting of a core and an ionic polymer.

In certain embodiments, the invention relates to any one of the composite nanoparticles described herein, wherein the ionic polymer comprises a plurality of repeat units having Formula II:

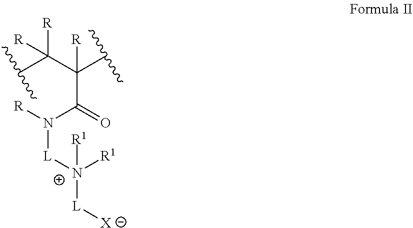

or a salt of either of them,

wherein, independently for each occurrence,

R is hydrogen or branched or unbranched, substituted or unsubstituted alkyl;

R¹ is hydrogen or branched or unbranched, substituted or unsubstituted alkyl;

L is branched or unbranched, substituted or unsubstituted alkylene, or substituted or unsubstituted arylene; and

X is —CO₂, —SO₃, —PO₃H, or —PO₂R.

In certain embodiments, the invention relates to any one of the composite nanoparticles described herein, wherein the ionic polymer comprises a plurality of repeat units having Formula II, a plurality of repeat units having Formula IIIa or Formula IVa, and a plurality of repeat units having Formula IIIb or Formula IVb:

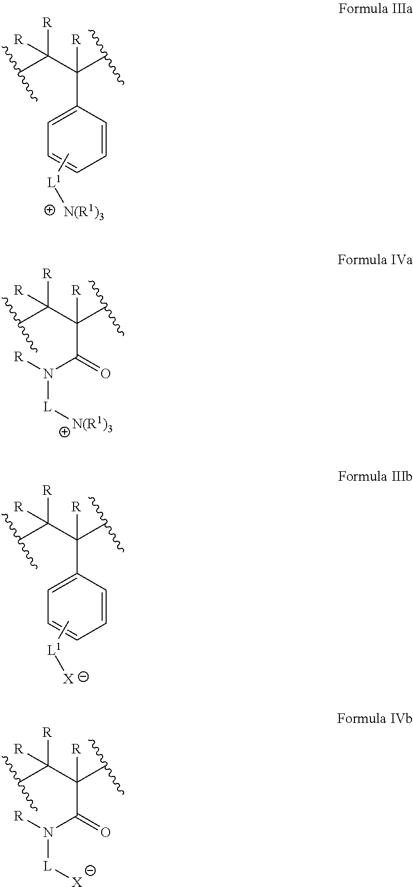

or a salt of any of them,

wherein, independently for each occurrence,

L¹ is absent, or a branched or unbranched, substituted or unsubstituted alkylene.

In certain embodiments, the invention relates to any one of the composite nanoparticles described herein, wherein the ionic polymer comprises a plurality of repeat units having Formula IIIa or Formula IVa, and a plurality of repeat units having Formula IIIb or Formula IVb:

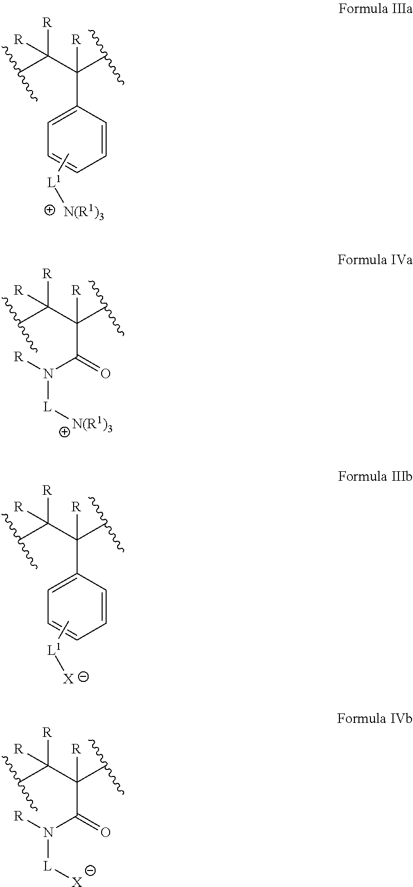

or a salt of any of them,

wherein, independently for each occurrence,

R is hydrogen or branched or unbranched, substituted or unsubstituted alkyl;

R¹ is hydrogen or branched or unbranched, substituted or unsubstituted alkyl;

L is branched or unbranched, substituted or unsubstituted alkylene, or substituted or unsubstituted arylene;

L¹ is absent, or a branched or unbranched, substituted or unsubstituted alkylene; and

X is —CO₂, —SO₃, —PO₃H, or —PO₂R.

In certain embodiments, the invention relates to any one of the composite nanoparticles described herein, wherein the ionic polymer further comprises a plurality of repeat units having Formula I:

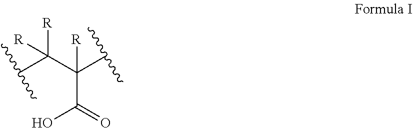

or a salt thereof.

In certain embodiments, the invention relates to any one of the composite nanoparticles described herein, wherein the core is selected from the group consisting of silica, carbon black, carbon nanotubes, graphene, iron, and magnetite.

In certain embodiments, the invention relates to an aqueous mixture comprising a plurality of composite nanoparticles as described herein.

In certain embodiments, the invention relates to any one of the aqueous mixtures described herein, wherein the composite nanoparticles form a suspension in the aqueous mixture.

In certain embodiments, the invention relates to any one of the aqueous mixtures described herein, wherein the average size of the nanoparticles in the suspension does not increase by more than about 30% after about 1 h.

In certain embodiments, the invention relates to a method of detecting the presence of, the concentration of, or the location of an analyte in a substrate, comprising the steps of:
contacting the substrate and a composition, wherein the composition comprises a plurality of reporter molecules at a first concentration, and a plurality of composite nanoparticles as described herein;
after a period of time, recovering a subset of the composite nanoparticles; and determining the concentration of the reporter molecules in the subset of composite nanoparticles.

### BRIEF DESCRIPTION OF THE FIGURES

FIG. 1 depicts a reaction scheme showing the hydrolysis at an elevated temperature of an exemplary ester-based polysulfobetaine, polysulfobetaine methacrylate.

FIG. 2 depicts a structural comparison of hindered amides and unhindered amides, where R, R₁, R₂, and R₃ are independently alkyl, aryl, diaryl, or other sterically bulky groups. The rate of hydrolysis of unhindered amides is greater than that of hindered amides.

FIG. 3 depicts the chemical structures of exemplary methacrylamide monomeric units of polyampholytes that possess chemical stability in water.

FIG. 4 depicts the chemical structures of exemplary methacrylamide monomeric units of polyampholytes that possess chemical stability in water.

FIG. 5 depicts the chemical structures of exemplary styrene monomeric units of polyampholytes that possess chemical stability in water.

FIG. 6 depicts the chemical structure of a polyampholyte comprising a random copolymer of acrylic acid and a zwitterionic monomer.

FIG. 7 depicts the chemical structure of a mixed polyampholyte, which has carboxylate moieties capable of chemical binding to the surface of nanoreporter particles.

FIG. 8 depicts the chemical structure of a mixed polyampholyte system having anchoring ability.

FIG. 9 depicts the chemical structure of a block copolymer polyampholyte system having anchoring ability.

FIG. 10 is a graph showing the colloidal stability over time of nanoparticles of the invention in low salinity Arab D brine.

FIG. 11 is a graph showing the colloidal stability over time of nanoparticles of the invention at 90 C in low salinity API brine.

FIG. 12 is a graph showing the colloidal stability over time of nanoparticles of the invention at 90 C in low salinity in API brine.

FIG. 13A depicts the structure of poly(sulfobetaine methacrylamide).

FIG. 13B depicts coil expansion in poly(sulfobetaine methacrylamide) via electrolyte and temperature modulation.

FIG. 14 depicts a schematic representation of responsive colloidal stabilization via polyzwitterion based graft polymer with increasing salinity and temperature. The polymer shell is shown to expand with addition of salt.

FIG. 15 has six panels (top left, top center, top right, bottom left, bottom center, bottom right) showing the SANS spectra (top) and partial Zimm plots (bottom) for 75K homopolymer with varying concentration of NaCl (left), concentration of CaCl₂ (center), and temperature (right).

FIG. 16 has six panels (top left, top center, top right, bottom left, bottom center, bottom right) showing the dependence of radius of gyration (top) and excluded volume parameter (bottom) for poly(SMBA) of different molecular weights (right) on salt type (NaCl, left; CaCl₂, center) and concentration, and on temperature. Higher molecular weight polymers are more sensitive to both size and shape change.

FIG. 17 has three panels (left, center, right) showing the dependence of Kuhn length on NaCl (left) and CaCl₂ (center) concentration, and on temperature (right) for different molecular weights of poly(SMBA).

FIG. 18 has three panels (left, center, right) showing the colloidal stability of MA/SBMA random copolymer functionalized silica nanoparticles in three different electrolyte environments (DI water, left; synthetic seawater, center; Arab D brine, right). No reversible clustering was observed, and measurements were discontinued beyond 30 days.

FIG. 19 has three panels (left, center, right) showing colloidal stability of MA/SBMA block copolymer functionalized silica nanoparticles in three different electrolyte environments at 90° C. (DI water, left; synthetic seawater, center; Arab D brine, right). No reversible clustering was observed, and measurements were discontinued beyond 30 days.

FIG. 20 has three panels (left, center, right) showing colloidal stability of MA/SBMA block copolymer functionalized polystyrene nanoparticles in three different electrolyte environments at 90° C. (DI water, left; synthetic seawater, center; Arab D brine, right).

FIG. 21 depicts GPC traces for poly(sulfobetaine methacrylamide) homopolymers in 0.5 M NaCl.

FIG. 22 depicts GPC Traces for poly(sulfobetaine methacrylamide)-b-(methacrylic acid) in 0.5 M NaCl.

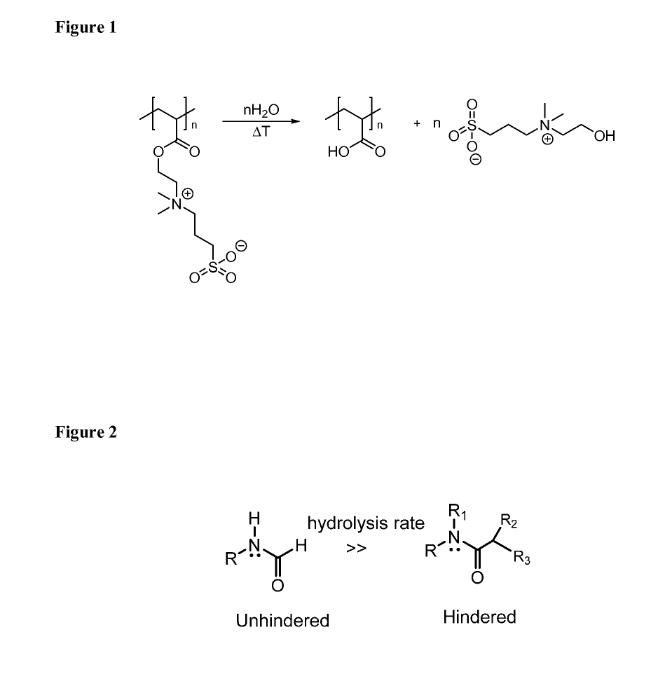

### DETAILED DESCRIPTION

### Overview

In certain embodiments, the invention relates to a composite nanoparticle, comprising a core and an ionic polymer. In certain embodiments, the core is a solid. In certain embodiments, the core is covalently attached to the ionic polymer. In certain embodiments, the invention relates to an aqueous mixture comprising a plurality of any one of the composite nanoparticles described herein. In certain embodiments, the aqueous mixture is a suspension. In certain embodiments, the aqueous mixture is stable over time. In certain embodiments, the composite nanoparticles do not aggregate in aqueous suspension at high salt concentrations and elevated temperatures for certain time periods.

In certain embodiments, the invention relates to any one of the composite nanoparticles described herein, wherein the ionic polymer is a polyampholyte. Polymers containing ionic groups can be divided into two groups: polyelectrolytes and polyampholytes. The former possess exclusively either anionic or cationic groups along the polymer chains, while the latter contain both anionic and cationic groups on the same or different monomer units within the polymer chain.

Properties of polyampholytes in solution are controlled by Columbic attraction between anionic and cationic groups. Polyampholytes can exhibit both polyelectrolyte and anti-polyelectrolyte behavior depending on the chemical structure and the composition of the polymer, the absence or presence of electrolytes, and solution pH. Anti-polyelectrolyte behavior refers to an increase in water solubility with the addition of salt. Anti-polyelectrolyte behavior leads to an increase in nanoreporter stability when polyampholytes are covalently attached to a nanoreporter core. Electrostatic stabilization of colloids is the process by which the attraction van der Waals forces are counterbalanced by the repulsive Coulomb forces acting between likewise-charged colloidal particles.

Polyampholytes can be categorized into four general classes based on the nature of their pendant functional groups, and charge variability in response to changes in pH and ionic concentration. First, type I polyampholytes are composed of strong cationic (i.e., quaternary alkyl ammonium groups) and strong anionic groups (i.e., sulfonate groups) which remain fully ionized over the entire range of pH or high salt concentrations. Type II polyampholytes feature strong cationic and weak anionic groups (e.g., carboxylate groups), the latter of which can be neutralized at low pH. Type III polyampholytes, contain weak cationic groups (e.g., amine hydrohalides) that can be neutralized at high pH, combined with strong anionic groups that remain charged over the whole range of pH. Finally, a type IV polyampholyte contains both weak anionic and weak cationic groups that are both responsive to changes in pH.

Type I polyampholytes retain their zwitterionic charge character over a wide range of pH, whereas the other classes will undergo transitions concomitant with pH or salt-induced charge neutralization of the weak cation or anion units. Water-soluble and water-swelling polyampholytes are used in a large number of applications including desalination of water, sewage treatment, flocculation, coagulation, drilling fluids, enhanced oil recovery, and catalysis. The oil recovery applications of polyampholytes include, but not limited to, rheology modification. See, for example, U.S. Pat. Nos. 4,637,882, 4,533,708, 4,460,758, 4,461,884, 4,540,496, 4,837,288, 5,068,278, 4,710,555, 4,640,945, 4,460,758, 5,132,285, 4,520,158, and 4,222,881, each of which is hereby incorporated by reference in its entirety.

In certain embodiments, the invention relates to any one of the composite nanoparticles described herein, wherein the ionic polymer does not comprise any ester functional groups. In certain embodiments, replacing ester functional groups with primary, secondary, or tertiary acrylamide groups may inhibit hydrolysis and stabilize the resultant nanoparticles. The low electrophilicity of an amide carbonyl group is reflected in its resistance to hydrolysis relative to functional groups such as esters. Furthermore, sterically hindered amides may be hydrolyzed more slowly than unhindered amides, due to their limited accessibility by nucleophiles, such as water and hydroxide ions (FIG. 2).

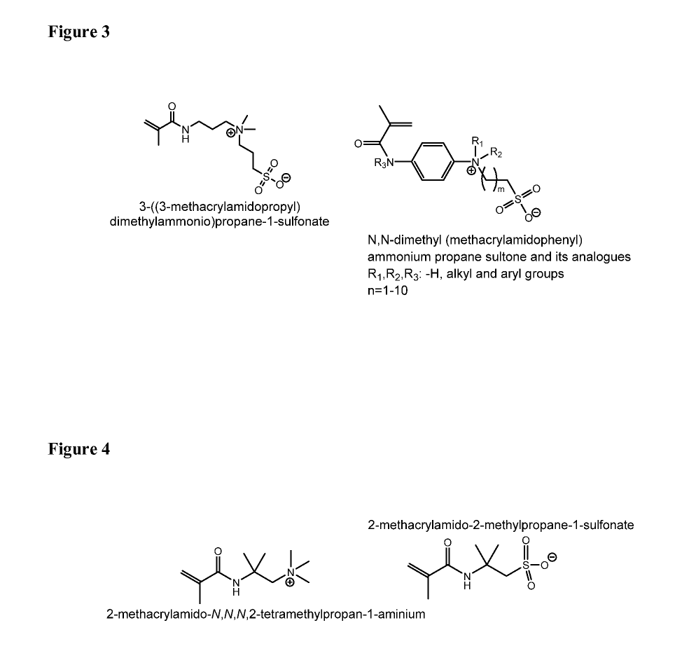

In certain embodiments, the invention relates to any one of the composite nanoparticles described herein, wherein the ionic polymer comprises repeat units derived from, for example, sulfobetaine methacrylamide or N,N-dimethyl (methacrylamidophenyl) ammonium propane sultone. Exemplary monomers are shown in FIG. 3.

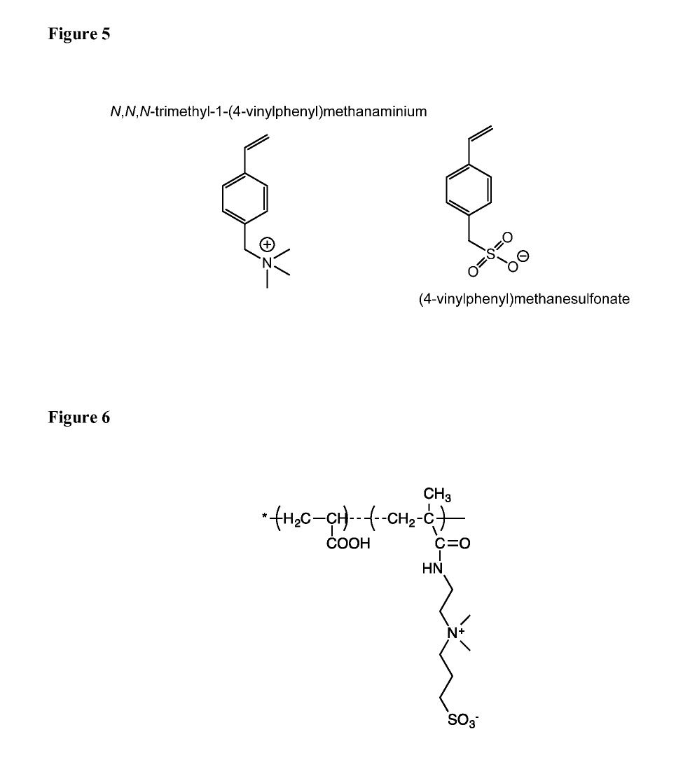

In certain embodiments, the invention relates to any one of the composite nanoparticles described herein, wherein the ionic polymer comprises repeat units derived from positively charged hindered methacrylamide-based monomers, negatively charged hindered methacrylamide-based monomers, positively charged styrene-based monomers, or negatively charged styrene-based monomers, wherein the molar ratio of positively charged repeat unit to negatively charged repeat unit in the ionic polymer is about 1:1. See, for example, FIG. 4 and FIG. 5.

In certain embodiments, the invention relates to a method of detecting the presence of or the location of a compound in a substrate, comprising the steps of: contacting the substrate with a plurality of any one of the composite nanoparticles described herein; and, after a period of time, collecting a subset of the plurality of composite nanoparticles.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The term “associated with” as used herein refers to the presence of either weak or strong or both interactions between molecules. For example weak interactions may include, for example, electrostatic, van der Waals, or hydrogen-bonding interactions. Stronger interactions, also referred to as being chemically bonded, refer to, for example, covalent, ionic, or coordinative bonds between two molecules. The term “associated with” also refers to a compound that may be physically intertwined within the foldings of another molecule, even when none of the above types of bonds are present. For example, an inorganic compound may be considered as being in association with a polymer by virtue of it existing within the interstices of the polymer.

The terms “comprise” and “comprising” are used in the inclusive, open sense, meaning that additional elements may be included.

The term “including” is used to mean “including but not limited to”. “Including” and “including but not limited to” are used interchangeably.

By the term “monomers compatible with” is meant those monomers which when used to provide repeating units in the polymer, provide units in amounts which do not interfere with the function of the cationic and anionic groups present or adversely affect the solubility of the resulting polyampholyte.

The term “polymer” is used to mean a large molecule formed by the union of repeating units (monomers). The term polymer also encompasses copolymers.

The term “co-polymer” is used to mean a polymer of at least two or more different monomers.

The term “particle size” is used to mean a number-average or weight-average particle size as measured by conventional particle size measuring techniques well known to those skilled in the art, such as dynamic or static light-scattering, sedimentation field-flow fractionation, photon-correlation spectroscopy, or disk centrifugation. By “an effective average particle size of less than about 1000 nm” it is meant that at least about 90% of the particles have a number-average or weight-average particle size of less than about 1000 nm when measured by at least one of the above-noted techniques.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

The term “alkylene” is art-recognized, and as used herein, pertains to a bidentate moiety obtained by removing two hydrogen atoms, either both from the same carbon atom, or one from each of two different carbon atoms, of a hydrocarbon compound, which may be aliphatic or alicyclic, or a combination thereof, and which may be saturated, partially unsaturated, or fully unsaturated. Examples of linear saturated C₁₋₁₀alkylene groups include, but are not limited to, —(CH₂)ₙ— where n is an integer from 1 to 10, for example, —CH₂— (methylene), —CH₂CH₂— (ethylene), —CH₂CH₂CH₂— (propylene), —CH₂CH₂CH₂CH₂— (butylene), —CH₂CH₂CH₂CH₂CH₂— (pentylene) and —CH₂CH₂CH₂CH₂CH₂CH₂— (hexylene). Examples of branched saturated C₁₋₁₀alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—. Examples of linear partially unsaturated C₁₋₁₀alkylene groups include, but are not limited to, —CH═CH— (vinylene), —CH═CH—CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—. Examples of branched partially unsaturated C₁₋₁₀alkylene groups include, but are not limited to, —C(CH₃)═CH—, —C(CH₃)═CH—CH₂—, and —CH═CH—CH(CH₃)—. Examples of alicyclic saturated C₁₋₁₀alkylene groups include, but are not limited to, cyclopentylene (e.g., cyclopent-1,3-ylene), and cyclohexylene (e.g., cyclohex-1,4-ylene). Examples of alicyclic partially unsaturated C₁₋₁₀alkylene groups include, but are not limited to, cyclopentenylene (e.g., 4-cyclopenten-1,3-ylene), and cyclohexenylene (e.g., 2-cyclohexen-1,4-ylene, 3-cyclohexen-1,2-ylene, and 2,5-cyclohexadien-1,4-ylene).

The term “aryl” is art-recognized, and includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The term “amido” is art recognized as an amino-substituted carbonyl and includes a moiety that may be represented by the general formula:

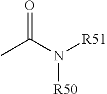

wherein R50 and R51 are as defined above. Certain embodiments of the amide in the present invention will not include imides which may be unstable.

The definition of each expression, e.g., alkyl, m, n, and the like, when it occurs more than once in any structure, is intended to be independent of its definition elsewhere in the same structure.

A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of the invention, the chemical elements are identified in accordance with the Periodic Table of the Elements, CAS version, Handbook of Chemistry and Physics, 67th Ed., 1986-87, inside cover.

### Exemplary Composite Nanoparticles

One aspect of the invention relates to a composite nanoparticle, comprising, consisting essentially of, or consisting of: a core; and an ionic polymer.

In certain embodiments, the invention relates to any one of the composite nanoparticles described herein, wherein the ionic polymer is a polyampholyte. In certain embodiments, the invention relates to any one of the composite nanoparticles described herein, wherein the polyampholyte is a polymeric sulfo- or carboxybetaine. Polymeric betaines comprise cationic moieties, such as a quaternary ammonium, and an anionic species, such as a sulfonate (sulfobetaines), a carboxylate (carbo- or carboxybetaines), a phosphate/phosphonate/phosphinate (phosphobetaines), or dicyanoethenolates.

In certain embodiments, the invention relates to any one of the composite nanoparticles described herein, wherein the ionic polymer comprises, consists essentially of, or consists of a plurality of repeat units having Formula II:

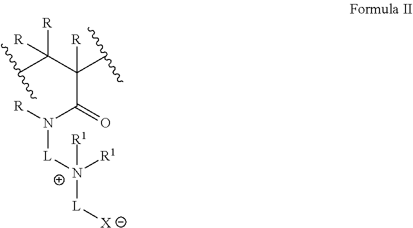

or a salt thereof,

wherein, independently for each occurrence,

R is hydrogen or branched or unbranched, substituted or unsubstituted alkyl;

R¹ is hydrogen or branched or unbranched, substituted or unsubstituted alkyl;

L is branched or unbranched, substituted or unsubstituted alkylene, or substituted or unsubstituted arylene; and

X is —CO₂, —SO₃, —PO₃H, or —PO₂R.

In certain embodiments, the invention relates to any one of the composite nanoparticles described herein, wherein the ionic polymer further comprises, consists essentially of, or consists of a plurality of repeat units having Formula IIIa or Formula IVa, and a plurality of repeat units having Formula IIIb or Formula IVb:

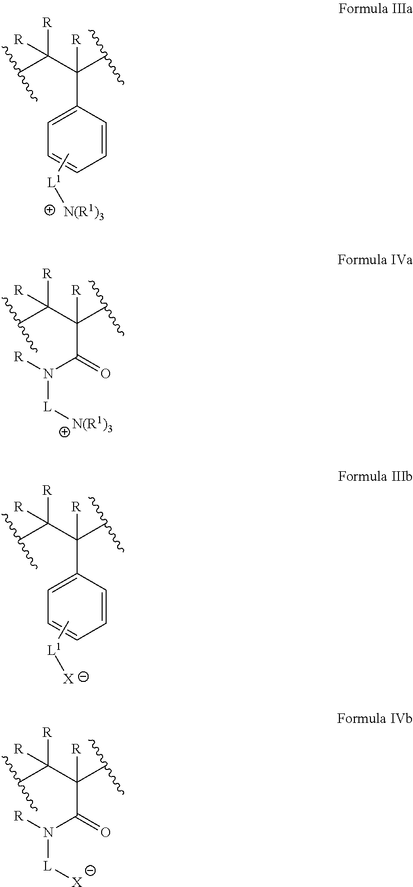

or a salt of any of them,

wherein, independently for each occurrence,

L¹ is absent, or a branched or unbranched, substituted or unsubstituted alkylene.

In certain embodiments, the invention relates to any one of the composite nanoparticles described herein, wherein the ionic polymer comprises, consists essentially of, or consists of a plurality of repeat units having Formula II, a plurality of repeat units having Formula IIIa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula II, a plurality of repeat units having Formula IIIa, and a plurality of repeat units having Formula IVb.

In certain embodiments, the invention relates to any one of the composite nanoparticles described herein, wherein the ionic polymer comprises, consists essentially of, or consists of a plurality of repeat units having Formula II, a plurality of repeat units having Formula IVa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula II, a plurality of repeat units having Formula IVa, and a plurality of repeat units having Formula IVb.

In certain embodiments, the invention relates to any one of the composite nanoparticles described herein, wherein the ionic polymer comprises, consists essentially of, or consists of a plurality of repeat units having Formula IIIa or Formula IVa, and a plurality of repeat units having Formula IIIb or Formula IVb:

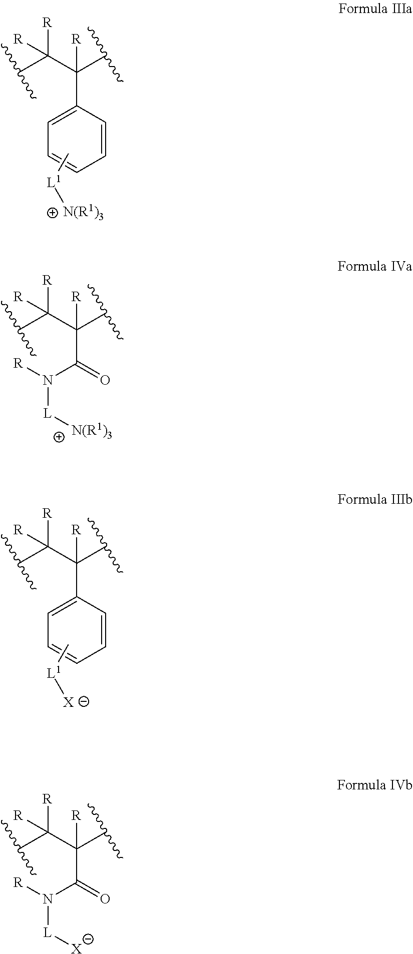

or a salt of any of them,

wherein, independently for each occurrence,

R is hydrogen or branched or unbranched, substituted or unsubstituted alkyl;

R¹ is hydrogen or branched or unbranched, substituted or unsubstituted alkyl;

L is branched or unbranched, substituted or unsubstituted alkylene, or substituted or unsubstituted arylene;

L¹ is absent, or a branched or unbranched, substituted or unsubstituted alkylene; and

X is —CO₂, —SO₃, —PO₃H, or —PO₂R.

In certain embodiments, the invention relates to any one of the composite nanoparticles described herein, wherein the ionic polymer comprises, consists essentially of, or consists of a plurality of repeat units having Formula IIIa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula IIIa, and a plurality of repeat units having Formula IVb.

In certain embodiments, the invention relates to any one of the composite nanoparticles described herein, wherein the ionic polymer comprises, consists essentially of, or consists of a plurality of repeat units having Formula IVa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula IVa, and a plurality of repeat units having Formula IVb.

In certain embodiments, the invention relates to any one of the composite nanoparticles described herein, wherein the ionic polymer further comprises a plurality of repeat units having Formula I:

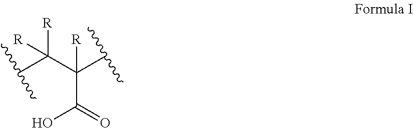

or a salt thereof,

wherein, independently for each occurrence,

R is hydrogen or branched or unbranched, substituted or unsubstituted alkyl.

In certain embodiments, the invention relates to any one of the composite nanoparticles described herein, wherein the ionic polymer comprises, consists essentially of, or consists of a plurality of repeat units having Formula I, a plurality of repeat units having Formula IIIa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula I, a plurality of repeat units having Formula IIIa, and a plurality of repeat units having Formula IVb.

In certain embodiments, the invention relates to any one of the composite nanoparticles described herein, wherein the ionic polymer comprises, consists essentially of, or consists of a plurality of repeat units having Formula I, a plurality of repeat units having Formula IVa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula I, a plurality of repeat units having Formula IVa, and a plurality of repeat units having Formula IVb.

In certain embodiments, the invention relates to any one of the composite nanoparticles described herein, wherein the ionic polymer comprises, consists essentially of, or consists of a plurality of repeat units having Formula I, a plurality of repeat units having Formula II, a plurality of repeat units having Formula IIIa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula I, a plurality of repeat units having Formula II, a plurality of repeat units having Formula IIIa, and a plurality of repeat units having Formula IVb.

In certain embodiments, the invention relates to any one of the composite nanoparticles described herein, wherein the ionic polymer comprises, consists essentially of, or consists of a plurality of repeat units having Formula I, a plurality of repeat units having Formula II, a plurality of repeat units having Formula IVa, and a plurality of repeat units having Formula IIIb.

In certain embodiments, the invention relates to any one of the composite nanoparticles described herein, wherein the ionic polymer comprises, consists essentially of, or consists of a plurality of repeat units having Formula I, a plurality of repeat units having Formula II, a plurality of repeat units having Formula IVa, and a plurality of repeat units having Formula IVb.

In certain embodiments, the invention relates to any one of the composite nanoparticles described herein, wherein the mole ratio of repeat units having Formula IIIa or Formula IVa to repeat units having Formula IIIb or Formula IVb in the ionic polymer is about 1:1.

In certain embodiments, the invention relates to any one of the composite nanoparticles described herein, wherein the mole ratio of repeat units of Formula I to repeat units of Formula II in the ionic polymer is from about 0.04:1 to about 2:1. In certain embodiments, the invention relates to any one of the composite nanoparticles described herein, wherein the mole ratio of repeat units of Formula I to repeat units of Formula II in the ionic polymer is about 0.04:1, about 0.06:1, about 0.08:1, about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about1.4:1, about 1.5:1, about 1.6:1, about 1.7:1, about 1.8:1, about 1.9:1, or about 2:1.

In certain embodiments, the invention relates to any one of the composite nanoparticles described herein, wherein the mole ratio of repeat units of Formula I to repeat units of Formula IIIa or Formula IVa in the ionic polymer is from about 0.04:1 to about 2.1. In certain embodiments, the invention relates to any one of the composite nanoparticles described herein, wherein the mole ratio of repeat units of Formula I to repeat units of Formula IIIa or Formula IVa in the ionic polymer is about 0.04:1, about 0.06:1, about 0.08:1, about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about1.4:1, about 1.5:1, about 1.6:1, about 1.7:1, about 1.8:1, about 1.9:1, or about 2:1.

In certain embodiments, the invention relates to any one of the composite nanoparticles described herein, wherein the mole ratio of repeat units of Formula I to repeat units of Formula IIIb or Formula IVb in the ionic polymer is from about 0.04:1 to about 2:1. In certain embodiments, the invention relates to any one of the composite nanoparticles described herein, wherein the mole ratio of repeat units of Formula I to repeat units of Formula IIIb or Formula IVb in the ionic polymer is about 0.04:1, about 0.06:1, about 0.08:1, about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about1.4:1, about 1.5:1, about 1.6:1, about 1.7:1, about 1.8:1, about 1.9:1, or about 2:1.

In certain embodiments, the invention relates to any one of the composite nanoparticles described herein, wherein the ionic polymer is a random copolymer.

In certain embodiments, the invention relates to any one of the composite nanoparticles described herein, wherein the ionic polymer is a block copolymer.

In certain embodiments, the invention relates to any one of the composite nanoparticles described herein, wherein the ionic polymer is a block copolymer having a first block consisting of a plurality of repeat units having Formula II. In certain embodiments, the invention relates to any one of the composite nanoparticles described herein, wherein the ionic polymer is a block copolymer having a first block consisting of a plurality of repeat units having Formula II; wherein the molecular weight of the first block is greater than about 40 kg/mol, greater than about 45 kg/mol, greater than about 50 kg/mol, greater than about 55 kg/mol, greater than about 60 kg/mol, greater than about 65 kg/mol, greater than about 70 kg/mol, greater than about 75 kg/mol, greater than about 80 kg/mol, greater than about 85 kg/mol, greater than about 90 kg/mol, greater than about 95 kg/mol, greater than about 100 kg/mol, greater than about 105 kg/mol, greater than about 110 kg/mol, greater than about 115 kg/mol, greater than about 120 kg/mol, or greater than about 125 kg/mol. In certain embodiments, the invention relates to any one of the composite nanoparticles described herein, wherein the ionic polymer is a block copolymer having a first block consisting of a plurality of repeat units having Formula II; wherein the molecular weight of the first block is from about 40 kg/mol to about 125 kg/mol, from about 50 kg/mol to about 125 kg/mol, from about 60 kg/mol to about 125 kg/mol, or from about 70 kg/mol to about 125 kg/mol. In certain embodiments, the invention relates to any one of the composite nanoparticles described herein, wherein the ionic polymer is a block copolymer having a first block consisting of a plurality of repeat units having Formula II; wherein the molecular weight of the first block is about 40 kg/mol, about 45 kg/mol, about 50 kg/mol, about 55 kg/mol, about 60 kg/mol, about 65 kg/mol, about 70 kg/mol, about 75 kg/mol, about 80 kg/mol, about 85 kg/mol, about 90 kg/mol, about 95 kg/mol, about 100 kg/mol, about 105 kg/mol, about 110 kg/mol, about 115 kg/mol, about 120 kg/mol, or about 125 kg/mol.

In certain embodiments, the invention relates to any one of the composite nanoparticles described herein, wherein the ionic polymer has from about 10 mol % to about 50 mol % repeat units having Formula IIIa; R is H; L¹ is —CH₂—; and R¹ is methyl.

In certain embodiments, the invention relates to any one of the composite nanoparticles described herein, wherein the ionic polymer has from about 10 mol % to about 50 mol % repeat units having Formula IIIb; R is H; L¹ is —CH₂—; and X is —SO₃.

In certain embodiments, the invention relates to any one of the composite nanoparticles described herein, wherein the ionic polymer has a molecular weight in the range of about 500 Da to about 250,000 Da. The polymers in the molecular weight ranges specified are soluble in water containing up to about 20 wt/v % NaCl or up to about 15 wt/v % CaCl², at a temperature from about 20° C. to about 90° C.

In certain embodiments, the invention relates to any one of the composite nanoparticles described herein, wherein R is hydrogen or methyl. In certain embodiments, the invention relates to any one of the composite nanoparticles described herein, wherein R is hydrogen. In certain embodiments, the invention relates to any one of the composite nanoparticles described herein, wherein R is methyl.

In certain embodiments, the invention relates to any one of the composite nanoparticles described herein, wherein R¹ is alkyl. In certain embodiments, the invention relates to any one of the composite nanoparticles described herein, wherein R¹ is methyl.

In certain embodiments, the invention relates to any one of the composite nanoparticles described herein, wherein L is branched alkylene. In certain embodiments, the invention relates to any one of the composite nanoparticles described herein, wherein L is unbranched alkylene. In certain embodiments, the invention relates to any one of the composite nanoparticles described herein, wherein L is unbranched, unsubstituted alkylene. In certain embodiments, the invention relates to any one of the composite nanoparticles described herein, wherein L is propylene. In certain embodiments, the invention relates to any one of the composite nanoparticles described herein, wherein L is ethylene. In certain embodiments, the invention relates to any one of the composite nanoparticles described herein, wherein L-X⁻ is —C(CH₃)₂—CH₂—X⁻. In certain embodiments, the invention relates to any one of the composite nanoparticles described herein, wherein L-N(R¹)₃⁺ is —C(CH₃)₂—CH₂—N(R¹)₃⁺.

In certain embodiments, the invention relates to any one of the composite nanoparticles described herein, wherein L is arylene. In certain embodiments, the invention relates to any one of the composite nanoparticles described herein, wherein L is phenylene. In certain embodiments, the invention relates to any one of the composite nanoparticles described herein, wherein L is 1,4-phenylene.

In certain embodiments, the invention relates to any one of the composite nanoparticles described herein, wherein X is —SO₃.

In certain embodiments, the invention relates to any one of the composite nanoparticles described herein, wherein L¹ is methylene.

In certain embodiments, the invention relates to any one of the composite nanoparticles described herein, wherein the ionic polymer is covalently bonded to the core.

In certain embodiments, the invention relates to any one of the composite nanoparticles described herein, wherein the ionic polymer is covalently bonded to the core via a linker. In certain embodiments, the invention relates to any one of the composite nanoparticles described herein, wherein the linker comprises an ester bond, an ether bond, or an amide bond. In certain embodiments, the invention relates to any one of the composite nanoparticles described herein, wherein the linker comprises an organosilane. In certain embodiments, the invention relates to any one of the composite nanoparticles described herein, wherein the linker comprises an amide bond derived from the reaction between the repeat units having Formula I in the ionic polymer and a free amine on the surface of the core. For example, the amide bond may be formed in the presence of dicyclohexylcarbodiimide (DCC) and dimethylaminopyridine (DMAP) in dimethylformamide (DMF) solvent.

In certain embodiments, the invention relates to any one of the composite nanoparticles described herein, wherein the core is solid.

In certain embodiments, the invention relates to any one of the composite nanoparticles described herein, wherein the core is selected from the group consisting of silica, carbon black, carbon nanotubes, graphene, iron, and magnetite.

In certain embodiments, the invention relates to any one of the composite nanoparticles described herein, wherein the composite nanoparticle has at least one dimension from about 10 nm to about 100 nm. In certain embodiments, the invention relates to any one of the composite nanoparticles described herein, wherein the composite nanoparticle is from about 10 nm to about 100 nm in all dimensions. In certain embodiments, the invention relates to any one of the composite nanoparticles described herein, wherein the composite nanoparticle is about 10 nm, about 20 nm, about 30 nm, about 40 nm, about 50 nm, about 60 nm, about 70 nm, about 80 nm, about 90 nm, or about 100 nm in one dimension, two dimensions, or all three dimensions.

In certain embodiments, the invention relates to any one of the composite nanoparticles described herein, further comprising a reporter molecule.

In certain embodiments, the invention relates to any one of the composite nanoparticles described herein, wherein the reporter molecule is non-covalently associated with the composite nanoparticle. Non-covalent associations may include, for example, ionic interactions, acid-base interactions, hydrogen bonding interactions, π-stacking interactions, van der Waals interactions, adsorption, physisorption, self-assembly and sequestration.

In certain embodiments, the invention relates to any one of the composite nanoparticles described herein, wherein the reporter molecule is a fluorescent dye, a lumenescent molecule, or a radioactive label.

In certain embodiments, the invention relates to any one of the composite nanoparticles described herein, wherein at least a portion of the reporter molecules is releasable from the composite nanoparticles upon exposure to an analyte. In other words, the plurality of reporter molecules is present in a first concentration in the composite nanoparticle prior to exposure, and in a second concentration after exposure.

### Exemplary Aqueous Mixtures

In certain embodiments, the invention relates to an aqueous mixture comprising a plurality of any one of the composite nanoparticles described herein.

In certain embodiments, the invention relates to any one of the aqueous mixtures described herein, wherein the aqueous mixture comprises NaCl. In certain embodiments, the invention relates to any one of the aqueous mixtures described herein, wherein the aqueous mixture comprises NaCl at a concentration of about 0.1 M, about 0.2 M, about 0.3 M, about 0.4 M, about 0.5 M, about 0.6 M, about 0.7 M, about 0.8 M, about 0.9 M, or about 1.0 M.

In certain embodiments, the invention relates to any one of the aqueous mixtures described herein, wherein the composite nanoparticles form a suspension in the aqueous mixture; the aqueous mixture comprises NaCl at a concentration of about 0.1 M, about 0.2 M, about 0.3 M, about 0.4 M, about 0.5 M, about 0.6 M, about 0.7 M, about 0.8 M, about 0.9 M, or about 1.0 M.

In certain embodiments, the invention relates to any one of the aqueous mixtures described herein, wherein the aqueous mixture comprises calcium chloride. In certain embodiments, the invention relates to any one of the aqueous mixtures described herein, wherein the aqueous mixture comprises calcium chloride at a concentration of about 0.1 M, about 0.2 M, about 0.3 M, about 0.4 M, about 0.5 M, about 0.6 M, about 0.7 M, about 0.8 M, about 0.9 M, or about 1.0 M.

In certain embodiments, the invention relates to any one of the aqueous mixtures described herein, wherein the aqueous mixture comprises magnesium chloride. In certain embodiments, the invention relates to any one of the aqueous mixtures described herein, wherein the aqueous mixture comprises magnesium chloride at a concentration of about 0.01 M, about 0.02 M, about 0.03 M, about 0.04 M, about 0.05 M, about 0.06 M, about 0.07 M, about 0.08 M, about 0.09 M, about 0.1 M, about 0.2 M, about 0.3 M, about 0.4 M, or about 0.5 M.

In certain embodiments, the invention relates to any one of the aqueous mixtures described herein, wherein the average size of the nanoparticles in the suspension does not increase by more than about 30%, more than about 20%, or more than about 10% after about 1 h, about 2 h, about 3 h, about 4 h, about 5 h, about 6 h, about 8 h, about 10 h, about 12 h, about 24 h, about 2 d, about 3 d, about 4 d, about 5 d, about 6 d, about 7 d, about 2 weeks, about 3 weeks, or about 4 weeks.

In certain embodiments, the invention relates to any one of the aqueous mixtures described herein, wherein, at a temperature of about 30° C., about 40° C., about 50° C., about 60° C., about 70° C., about 80° C., about 90° C., about 100° C., about 110° C., about 120° C., about 130° C., about 140° C., or about 150° C., the average size of the nanoparticles in the suspension does not increase by more than about 30%, more than about 20%, or more than about 10% after about 1 h, about 2 h, about 3 h, about 4 h, about 5 h, about 6 h, about 8 h, about 10 h, about 12 h, about 24 h, about 2 d, about 3 d, about 4 d, about 5 d, about 6 d, about 7 d, about 2 weeks, about 3 weeks, or about 4 weeks.

### Exemplary Methods

In certain embodiments, the invention relates to a method of detecting the presence of, the concentration of, or the location of an analyte in a substrate, comprising the steps of:
contacting the substrate and a composition, wherein the composition comprises a plurality of reporter molecules at a first concentration, and a plurality of any one of the composite nanoparticles described herein;
after a period of time, recovering a subset of the composite nanoparticles; and
determining the concentration of the reporter molecules in the subset of composite nanoparticles.

In certain embodiments, the invention relates to any one of the methods described herein, wherein at least a portion of the reporter molecules is releasable from the composite nanoparticles upon exposure to the analyte. In other words, the plurality of reporter molecules is present in a first concentration in the composition prior to exposure, and in a second concentration after exposure. The methods further include assaying the composition to determine the second concentration. In further embodiments, the methods also include assaying the liquid medium for the portion of the plurality of reporter molecules released from the composite nanoparticles.

In certain embodiments, the invention relates to any one of the methods described herein, wherein the reporter molecule is non-covalently associated with the composite nanoparticle. Non-covalent associations may include, for example, ionic interactions, acid-base interactions, hydrogen bonding interactions, π-stacking interactions, van der Waals interactions, adsorption, physisorption, self-assembly and sequestration. In certain embodiments, the reporter molecule is a fluorescent dye, a lumenescent molecule, or a radioactive label.

In certain embodiments, the invention relates to any one of the methods described herein, wherein the substrate is a geological formation, such as, for example, an oilfield or an oil well. In general, in methods for assaying a geological formation, the compositions are released downhole via injection, which is followed by injection of water or brine. The compositions move through the geological formation until the water or brine injection terminates. After a period of time the flow is reversed, such that the compositions are then pulled back through the injection well or a production well for analysis. Samples are collected and analyzed by standard characterization techniques. The residence time of the compositions in the geological formation is dependent on a number of factors including, for example, the period of time before the flow is reversed, and the distance the compositions initially travel during injection. During their time in the geological formation, the compositions lose hydrophobic reporter molecules to any hydrophobic media contained therein, such as, for example, petroleum. Given the residence time, as well as the known temperature, the amount of hydrophobic reporter molecules lost can be diagnostic of the amount of petroleum contained in the geological formation.

In certain embodiments, the invention relates to any one of the methods described herein, wherein the substrate is a liquid medium, such as an aqueous salt solution. Aqueous salt solutions, such as brine, are commonly encountered in geological formations, particularly those used for oil production. In certain embodiments, the invention relates to any one of the methods described herein, wherein the substrate is a surface water source, a groundwater source, or a wastewater source. In various embodiments, the liquid medium is flowing. In some embodiments, the liquid medium is adsorbed on to a solid surface, such as, for example, a rock surface.

In certain embodiments, the invention relates to any one of the methods described herein, wherein the analyte is an organic compound, an inorganic compound, an ion, or a heavy metal. In another embodiment, the analyte may be a physical parameter, including, for example, pressure, temperature, pH, redox potential and conductivity.

The transport of hydrophobic organic molecules through porous media, such as, for example, soil has been studied for many years to help understand the percolation of pollutants into the environment. In isolation, hydrophobic organic molecules adsorb very strongly to nearly all types of soil. However, hydrophobic organic molecules disperse much more broadly in the environment than would be expected given their strong affinity for binding to soil. One possible explanation for this behavior is that organic macromolecules having amphiphilic characteristics may sequester small hydrophobic organic molecules and facilitate their transport by carrying them within the organic macromolecule. This effect has been demonstrated in the laboratory with amphiphilic molecules, such as, for example, cyclodextrin, which shows highly efficient transport of hydrophobic molecules. Embodiments described herein demonstrate compositions and methods for selective transport and release of both non-covalently adsorbed and covalently bonded reporter molecules from water- and brine-soluble nanomaterials. By analyzing the compositions after release or uptake of reporter molecules, various inferences can be made regarding the environment to which the compositions have been exposed.

In some embodiments, the invention relates to any one of the methods described herein, wherein the analyte is a hydrophobic substance, such as, for example, petroleum. In other embodiments, the analyte is a sulfur-containing compound, such as, for example, hydrogen sulfide or thiols.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Polymer Synthesis

This Example illustrates synthesis of a random copolymer from acrylic acid and a zwitterionic monomer.

[3-(Methacryloylamino)propyl]dimethyl(3-sulfopropyl)ammonium hydroxide inner salt (7.12 grams), acrylic acid (0.384 mL) and 0.075 grams of potassium persulfate were dissolved in 22 mL of deionized water in a round bottom flask. The flask was deaerated by nitrogen bubbling through the reaction vessel for 30 min, and the reactor was heated to 80° C. The reaction was allowed to continue for 6 h, after which it was terminated via exposure to air. The polymer was precipitated with acetone, and dried via vacuum. The structure of the random copolymer is shown in FIG. 6.

### Example 2

### Polymer Synthesis

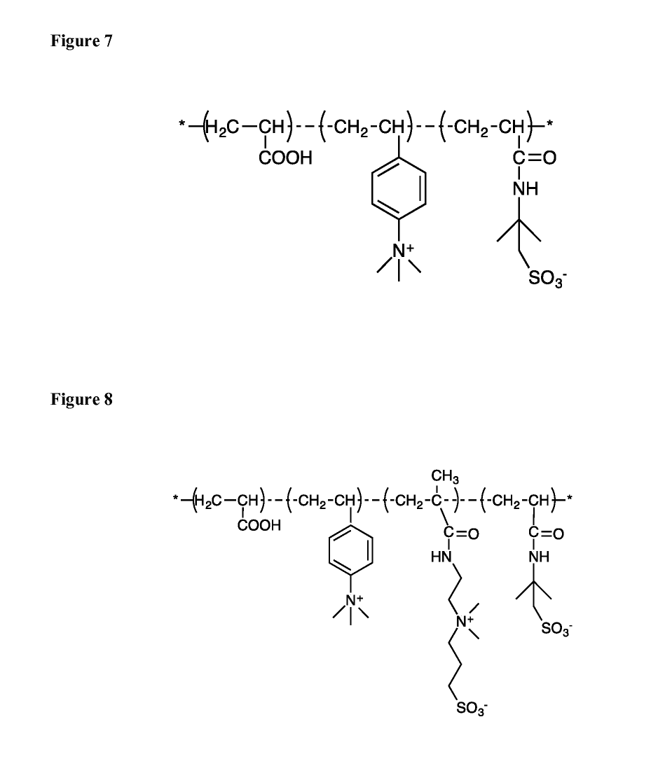

4.2 mL of 2-acrylamido-2-methylpropane sulfonate (50 aqueous weight %), 2.318 g of (vinylbenzyl)trimethylammonium chloride, 0.384 mL of acrylic acid, and 0.075 g of potassium persulfate were dissolved in water to a volume of 22 mL in a round bottom flask. Nitrogen was bubbled through the reaction vessel for 30 minutes, and the reactor was heated to 80° C. The reaction was allowed to continue for 6 hours, after which it was terminated via exposure to air. The polymer was precipitated with acetone, and dried via vacuum. See FIG. 7.

### Example 3

### Polymer Synthesis

1.62 g of [3-(methacryloylamino)propyl] dimethyl(3-sulfopropyl)ammonium hydroxide inner salt, 1.736 g of (vinylbenzyl)trimethylammonium chloride, 3.17 mL of 2-acrylamido-2-methylpropane sulfonate (50 aqueous weight %), 0.384 mL of acrylic acid, and 0.075 g of potassium persulfate were dissolved in water to a volume of 22 mL in a round bottom flask. Nitrogen was bubbled through the reaction vessel for 30 minutes, and the reactor was heated to 80° C. The reaction was allowed to continue for 6 hours, after which it was terminated via exposure to air. The polymer was precipitated with acetone, and dried via vacuum. See FIG. 8.

### Example 4

### Polymer Synthesis

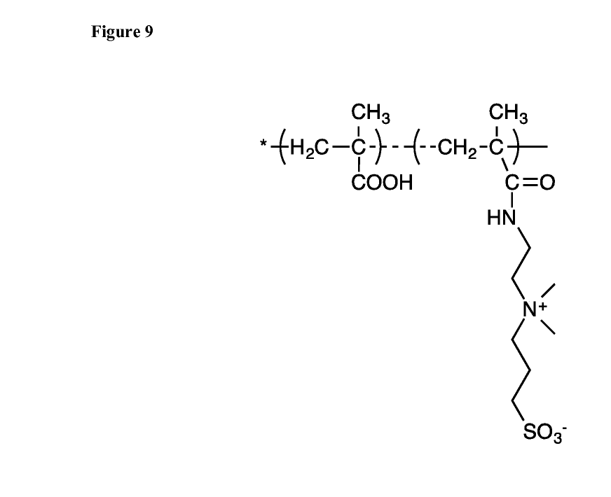

5.84 g of [3-(methacryloylamino)propyl]dimethyl(3-sulfopropyl)ammonium hydroxide inner salt, 0.0331 g of 4-cyano-4-(phenylcarbonothioylthio)pentanoic acid, and 0.0112 g of 4,4′-azobis(4-cyanovaleric acid) were dissolved in water to a volume of 13,3 mL in a round bottom flask. Nitrogen was bubbled through the reaction vessel for 30 minutes, and the reactor was heated to 60° C. The reaction was allowed to continue for 9 hours, after which 0.328 mL of degassed methacrylic acid was added to the reaction mixture. The reaction was allowed to continue for another 4 hours, after which it was terminated by exposing to air, See FIG. 9.

### Example 5

### Silica Functionalization

2 mL of Ludox TM-40 (40 wt % colloidal silica suspension in water) was washed with 50 mL of ethanol and subsequently centrifuged at 7000 RPM. The washing and centrifugation process was repeated to obtain 20 nm silica nanoparticles dispersed in ethanol. This dispersion was treated with 0.300 mL of aminopropyltrimethoxysilane at 30° C. overnight to obtain amine decorated nanoparticles. The nanoparticles were separated from excess aminopropyltrimethoxysilane via two ethanol washing and centrifugation cycles.

### Example 6

### Polyampholyte Grafting

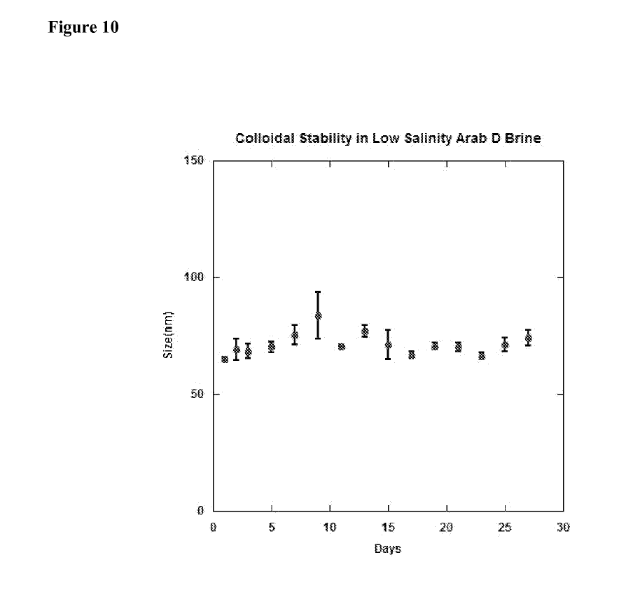

1 g of the polymer from Example 1 was dissolved in 15 mL of water. Separately, 0.1 g of amine-decorated silica from Example 5 was dispersed via sonication in 5 mL of water. The nanoparticle dispersion was drop-by-drop added to the polymer solution, while stirring at 30° C. Subsequently, 0.12 g of 1-ethyl-3-[3-dimethylaminopropyl]carbodiimide hydrochloride, and 0.60 g of N-hydroxysuccinimide were added to the reaction mixture. The pH was adjusted to 4.75 and the reaction was allowed to continue overnight. The nanoparticles were centrifuged and washed twice with 1 M NaCl water, and finally dispersed in brine (for example, API (2 wt % CaCl₂ and 8 wt % NaCl) or Low Salinity Arab-D Brine (comprising an aquepous solution of NaCl (74.19 g/L), CaCl₂.2H₂O (49.79 g/L), MgCl₂.6H₂O (13.17 g/L), BaCl₂ (0.01 g/L), Na₂SO₄ (0.60 g/L), and NaHCO₃ (0.51 g/L))). For illustrative purposes, the long-term stability of the polymer-functionalized nanoparticles in API brine and Low Salinity Arab-D Brine are shown in FIG. 10.

### Example 7

### Polyampholyte Grafting

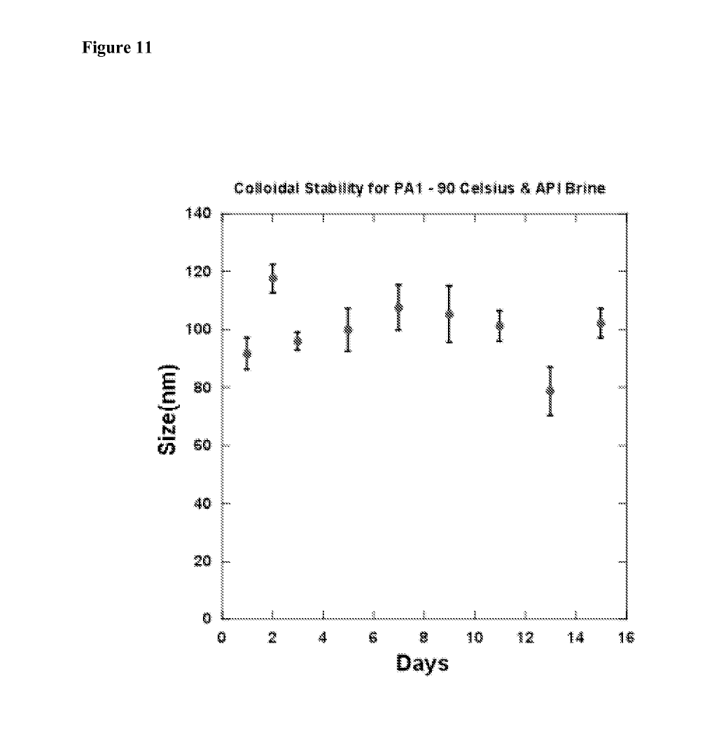

1 g of the polymer from Example 2 was dissolved in 15 mL of water. Separately, 0.1 g of amine-decorated silica from Example 5 was dispersed via sonication in 5 mL of water. The nanoparticle dispersion was drop-by-drop added to the polymer solution, while stirring at 30° C. Subsequently, 0.12 g of 1-ethyl-3-[3-dimethylaminopropyl]carbodiimide hydrochloride, and 0.60 g of N-hydroxysuccinimide were added to the reaction mixture. The pH was adjusted to 4.75 and the reaction was allowed to continue overnight. The nanoparticles were centrifuged and washed twice with 1 M NaCl water, and finally dispersed in brine (for example, API or Low Salinity Arab-D Brine). For illustrative purposes, the long-term stability of the polymer-functionalized nanoparticles in API brine and Low Salinity Arab-D Brine are shown in FIG. 11.

### Example 8

### Polyampholyte Grafting

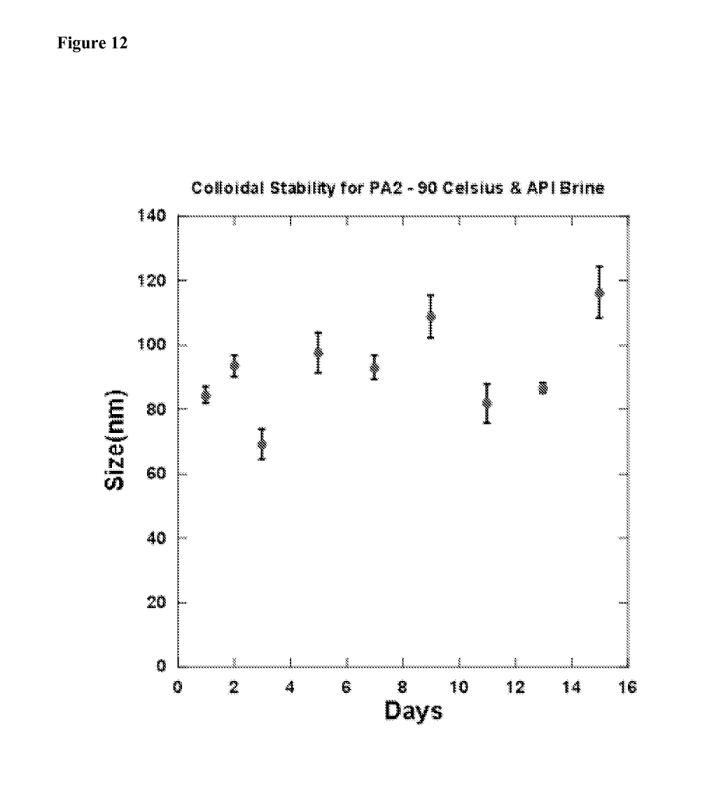

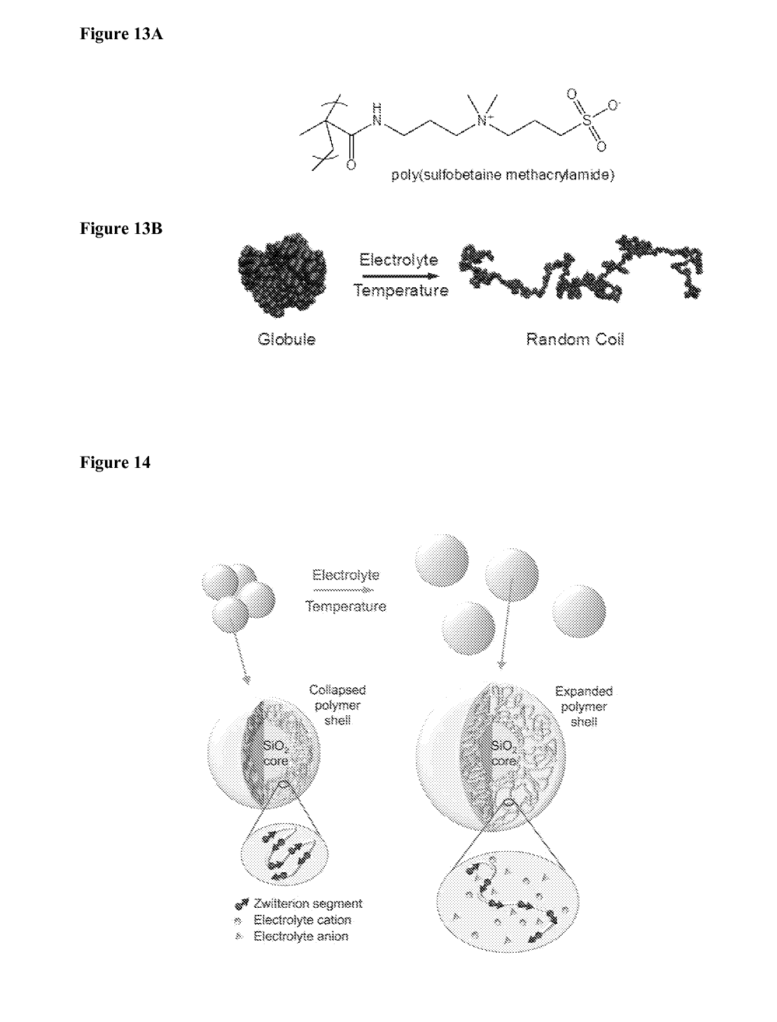

1 g of the polymer from Example 3 was dissolved in 15 mL of water. Separately, 0.1 g of amine-decorated silica from Example 5 was dispersed via sonication in 5 mL of water. The nanoparticle dispersion was drop-by-drop added to the polymer solution, while stirring at 30° C. Subsequently, 0.12 g of 1-ethyl-3-[3-dimethylaminopropyl]carbodiimide hydrochloride, and 0.60 g of N-hydroxysuccinimide were added to the reaction mixture. The pH was adjusted to 4.75 and the reaction was allowed to continue overnight. The nanoparticles were centrifuged and washed twice with 1 M NaCl water, and finally dispersed in brine (for example, API or Low Salinity Arab-D Brine). For illustrative purposes, the long-term stability of the polymer-functionalized nanoparticles in API brine and Low Salinity Arab-D Brine are shown in FIG. 12.

### Example 9

### Materials and Methods

Methacrylic acid (99%) (MA), SBMA (96%), 4,4’-azobis(4-cyanovaleric acid) (ACPA), (3-aminopropyl)trimethoxysilane (97%) (APTMS), N-(3-dimethylaminopropyl)-N′-ethylcarbodiimide hydrochloride (crystalline) (EDC), N-hydroxysuccinimide (98%) (NHS) and Ludox AS-40 Silica were purchased from Sigma Aldrich USA and used without further purification. The chain transfer agent 4-cyano-4 phenylcarbonothioylthio)pentatonic acid (CPP) was purchased from Strem Chemicals and used without further purification. Amine functionalized polystyrene nanoparticles (28 nm) were purchased from Life Technologies USA. Salts were purchased from Sigma Aldrich USA at the following purity levels: NaCl (99%), CaCl₂.6H₂O (98%), MgCl₂.6H₂O (99%), Na₂SO₄ (99%), NaHCO₃ (99.7%), KCl (99%), KBr (99%). A 1260 Agilent gel permeation chromatograph was used to determine the molecular weight distribution in combination with a Wyatt miniDAWN TREOS for static light scattering. A Brookhaven 90Plus PALS was used for dynamic light scattering and a CONTIN algorithm was used to analyse autocorrelation functions. While all samples were long-term heat aged at 90° C., measurements were made up to 65° C. due to machine limitations. Small-angle neutron scattering measurements were done at the National Institute of Standards and Technology (NIST) in Gaithersburg, Md.

### Polymer Synthesis

Homopolymer: Molar ratios were varied from 50:1:0.25 to 400:1:0.25 ([M]:[I]:[CTA]) to tune the degree of polymerization. Initial monomer concentration was maintained at 1.5 M. A typical synthesis was as follows: 20 mM of SBMA, 0.4 mM of CPP, and 0.1 mM of ACPA were dissolved in 0.5 M NaCl to a volume of 13.3 mL in a round bottom flask. Nitrogen was bubbled through the solution for 30 minutes, and the reactor was heated to 60° C. The polymerization was terminated after 24 hours by exposure to atmospheric oxygen. The product was recovered via precipitation in acetone.

Random Copolymer: 25 mM of SBMA, 5 mM of MA and 0.3 mM of potassium persulfate were dissolved in 22 mL of deionized water in a round bottom flask. Nitrogen was bubbled through the solution for 30 minutes, and the reactor was heated to 80° C. The polymerization was terminated after 6 hours, by exposure to atmospheric oxygen. The shorter reaction time relative to that used for the homopolymer was due to the higher temperature at which the polymerization was conducted. The product was recovered via precipitation in acetone.

Block Copolymer: A small MA block (anchor block of 20 units) was synthesized and used as a macromolecular chain transfer agent for subsequent polymerization of an SBMA block of varying lengths.

Anchor Block: 10 mM of methacrylic acid and 0.5 mM of CPP were dissolved in 6.9 mL of isopropanol in a 25 mL round bottom flask. 0.1 mM of ACPA was dissolved in 0.5 mL of methanol and added to the solution. Nitrogen was bubbled through the solution for 30 minutes, and the mixture was heated to 60° C. The polymerization was again terminated after 24 hours by exposure to atmospheric oxygen. The resulting solution was noted to be moderately viscous. The polymer was precipitated with diethyl ether.

Stabilizer Block: Molar ratios were varied from 50:1:0.25 to 400:1:0.25 ([M]:[I]:[CTA]) respectively to tune the degree of polymerization. Initial monomer concentration was maintained at 1.5 M.

An example synthesis was as follows: 20 mM of SBMA, 0.4 mM of anchor block polymer, and 0.1 mM ACPA were dissolved in 0.5 M NaCl to a volume of 13.3 mL in a round bottom flask. Nitrogen was bubbled through the solution for 30 minutes, and the reactor was heated to 60° C. The polymerization was terminated after 24 hours by exposure to atmospheric oxygen.

### Small-Angle Neutron Scattering

SANS measurements were performed on the NG-3 (30 m) beamline_at the National Institute of Standards and Technology (NIST) in Gaithersburg, Md. Neutrons with a wavelength of 6 Å were selected, and three sample detector distances were used to probe a range of wave vectors between 0.004 and 0.4 Å⁻¹. Data normalization using accepted procedures gave the absolute cross section 1(Q) (cm⁻¹) as a function of momentum transfer Q (Å⁻¹). Samples were prepared using D₂O (scattering length density ρ=6.33×10¹⁰ cm⁻²) to provide the necessary contrast and were placed in Hellma fused silica cuvettes of path length 2 mm. A concentration of 2.5% (w/w) was chosen for all the samples. Data were analysed in absolute units (I(Q)/cm⁻¹) with fitted scale factors consistent with expectations based on sample compositions. Data were analysed via the polymer excluded volume (PEV) model, as well as via Porod and Zimm approximations.

### Nanoparticle Functionalization

Amine Functionalization of Silica Nanoparticles: 2 g of Ludox TM-40 was washed with 50 ml of ethanol and subsequently centrifuged at 16,128 RCF for 30 minutes. The washing and centrifugation process was repeated to obtain 20 nm silica nanoparticles dispersed in ethanol. This dispersion was treated with 0.300 mL of APTMS at 30° C. overnight to obtain amine-decorated nanoparticles. The nanoparticles were separated from excess APTMS by two ethanol washing and centrifugration cycles. The particles were finally dispersed in 40 mL of water at pH 5 for a concentration of 2% w/v.

Polymer Grafting: 3 g of synthesized block copolymer was dissolved in 15 mL of water, and 2.5 mL of amine-functionalized silica/polystyrene was added dropwise with stirring at 30° C. Subsequently, 0.6 g of EDC was added to the reaction mixture. The pH was adjusted to 4.5 and the reaction was allowed to continue overnight. The polymer-functionalized nanoparticles were separated from free polymer via centrifugation, and finally dispersed in the brine of choice at a concentration of 0.5% w/v. (DI Water, Synthetic seawater, Arab D brine, defined in Table 1).

TABLE 1
Brine Compositions

| | Synthetic | Arab D |
|---|---|---|
| Salt | Seawater (g/L) | Brine (g/L) |
| NaCl | 24 | 74 |
| CaCl₂•6H₂O | 2.0 | 49 |
| MgCl₂•6H₂O | 11 | 13 |
| Na₂SO₄ | 4.0 | 0.60 |
| NaHCO₃ | — | 0.50 |
| KCl | 0.70 | — |
| KBr | 0.10 | — |
| TDS mg/dm³ | 57,269 | 120,000 |

### Results and Discussion

Polybetaines are a unique subclass of polyampholytes that exhibit distinctly different properties from those of conventional anionic or cationic polyelectrolytes. In contrast to most polyampholytes, whose spatial distribution of cationic and anionic groups is typically random, polybetaines bear an equal number of cationic (quaternary ammonium) and anionic (sulfonate) groups whose spatial distribution is defined monomerically.

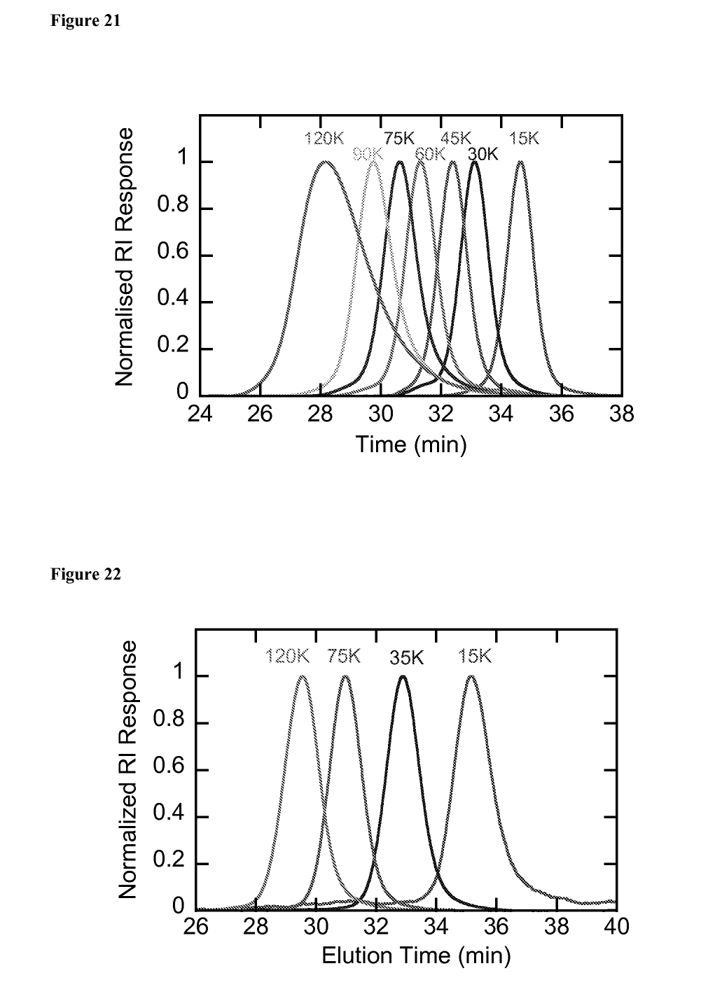

Low polydispersity poly(SBMA) was synthesized using CPP as the chain transfer agent. Chain lengths of 50, 100, 150, 200, 250, 300 and 400 were targeted, with excellent control over the entire range of polymerization conditions. A slight loss of living character was observed at the higher chain lengths, but was mostly avoided by ensuring appropriate conditions to prevent aminolysis and hydrolysis of the chain transfer agent. Polydispersities are reported in Table 2, and overlayed chromatograms are shown in FIG. 21.

TABLE 2
Poly(SBMA) Molecular Weight and Polydispersity
Synthesized via RAFT Polymerization

| Sample | Mₙ (g/mol) | Polydispersity |
|---|---|---|
| 15K | 15,680 | 1.006 |
| 30K | 31,100 | 1.026 |
| 45K | 47,810 | 1.070 |
| 60K | 53,650 | 1.126 |
| 75K | 70,890 | 1.092 |
| 90K | 89,891 | 1.120 |
| 120K | 124,000 | 1.235 |

### Small-Angle Neutron Scattering

The antipolyelectrolyte phenomenon was probed via small-angle neutron scattering (SANS), which was used to determine the radii of gyration (R_{g}) and excluded volume parameters (v) for 15K, 45K, 75K and 120K homopolymers under the extreme conditions of salinity and temperatures representative of subsurface reservoirs. The phase behaviour of polybetaines has been accepted to follow a simple Flory-Huggins model:

K_{B}T χ=εₘₛ−0.5[εₘₘ+εₛₛ] (2)

where χ is the interaction parameter and εₘₛ, εₘₘ and εₛₛ are the monomer-solvent, monomer-monomer, and solvent-solvent contact energies respectively. Contact energies are sensitive to the Debye length, and solvent quality, which can be modulated through the electrolyte concentration and temperature. The effects of these solution conditions on polymer conformation are particularly evident in log-log plots in the Porod regime (0.02<Q<0.05), from which the fractal dimension of the polymer can be obtained via the equation:

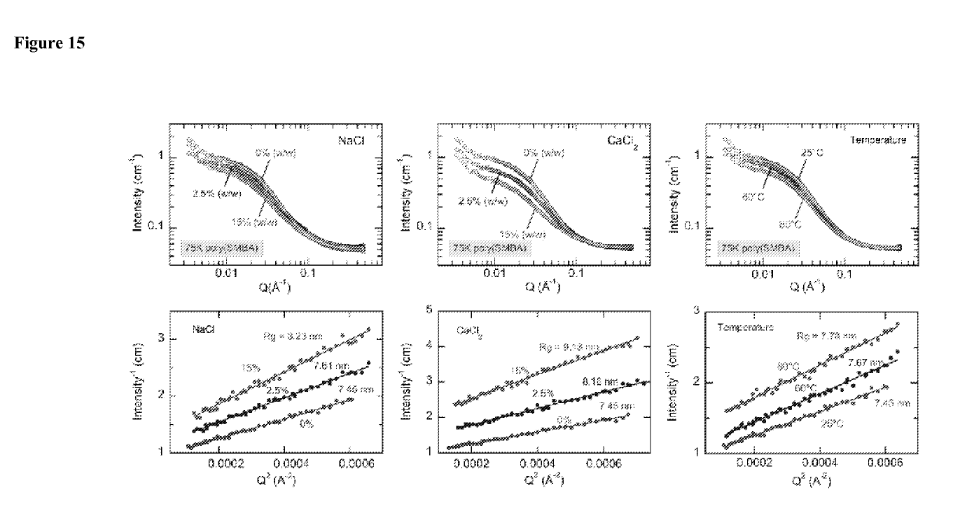

log I=log A−n log Q (3)

where n, the Porod slope, is the inverse of the excluded volume parameter v (i.e. n=1/v). A slope of n=3 is the signature of a globular coil, while a slope of n=5/3 represents a fully swollen coil. Therefore the magnitude of n is expected to decrease with improvements in solvent quality. This trend was indeed observed and example log-log plots for the 75K homopolymer are shown in FIG. 15; Porod fit parameters are tabulated in Tables 3-5.

TABLE 3
Comparison of R_{g} - NaCl
NaCl

| Concentration (weight %) | Porod | PEV |
|---|---|---|
| 0 | 0.41 | 0.41 |
| 2.5 | 0.52 | 0.53 |
| 15 | 0.53 | 0.56 |

TABLE 4
Comparison of R_{g} - CaCl₂
CaCl₂

| Concentration (weight %) | Porod | PEV |
|---|---|---|
| 0 | 0.41 | 0.41 |
| 2.5 | 0.51 | 0.51 |
| 15 | 0.55 | 0.55 |

TABLE 5
Comparison of R_{g} - Temperature
Temperature

| Celsius | Porod | PEV |
|---|---|---|
| 25 | 0.41 | 0.41 |
| 60 | 0.42 | 0.43 |
| 80 | 0.48 | 0.47 |

The excluded volume parameter was observed to be v˜0.41 with no added salt, at a temperature of 25° C, and increased to a maximum value of v˜0.56 upon addition of NaCl or CaCl₂. Temperature was noted to have a similar but weaker effect, with v increasing from ˜0.41 to ˜0.48 over the range examined. Additionally, partial Zimm plots were used to quantify changes in R_{g} as a function of electrolyte concentration, type and temperature. A Lorentzian form for the Q dependence of scattering intensity was assumed:

$$I ( Q ) = \frac{{I}_{o}}{1 + {Q}^{2} {ξ}^{2}} \qquad ( 4 )$$

In the low Q region, ξ=R_{g}/\sqrt{3}, and a plot of I⁻¹ vs Q² allows the correlation length ξ to be estimated. Partial Zimm plots for the 75K homopolymer are shown in FIG. 15, and the results are tabulated in Tables 6-8.

TABLE 6
Comparison of R_{g} - NaCl
NaCl

| Concentration (weight %) | Zimm | PEV |
|---|---|---|
| 0 | 7.45 | 6.63 |
| 2.5 | 7.61 | 7.42 |
| 15 | 8.23 | 7.98 |

TABLE 7
Comparison of R_{g} - CaCl₂
CaCl₂

| Concentration (weight %) | Zimm | PEV |
|---|---|---|
| 0 | 7.45 | 6.63 |
| 2.5 | 8.16 | 7.16 |
| 15 | 9.13 | 7.94 |

TABLE 8
Comparison of R_{g} - Temperature
Temperature

| Celsius | Zimm | PEV |
|---|---|---|
| 25 | 7.45 | 6.63 |
| 60 | 7.67 | 6.86 |
| 80 | 7.78 | 6.90 |

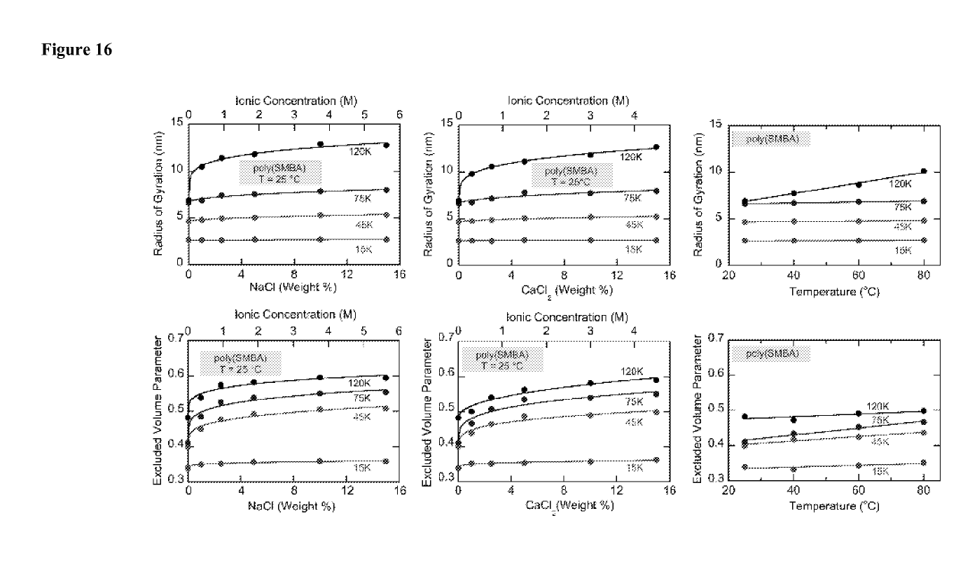

A polymer excluded volume model with form factor:

$$P ( Q ) = 2 \int ( 1 - x ) exp [ - \frac{{Q}^{2} {a}^{2}}{6} {N}^{2 v} {x}^{2 v} ] \qquad ( 5 )$$

where a is the chain statistical segment (Kuhn) length and N is degree of polymerization, was also used to fit the scattering profiles for all four molecular weight polymers across the entire range of temperatures and salt concentrations examined (model fits are shown as light lines on the spectra in FIG. 15). The extracted model parameters were in excellent agreement with the Porod and partial Zimm fits (comparisons in Tables 3-8), and the dependence of R_{g} and v on solution conditions is shown in FIG. 16 and tabulated in Tables 9-14.

TABLE 9
R_{g} variation with NaCl

| | |
|---|---|
| Salt | |
| Concentration | |
| (weight %) | Radius of Gyration (nm) |

| NaCl | 15K | 45K | 75K | 120K |
|---|---|---|---|---|
| 0 | 2.65 | 4.67 | 6.63 | 6.91 |
| 1 | 2.64 | 4.79 | 6.89 | 10.46 |
| 2.5 | 2.61 | 4.93 | 7.42 | 11.42 |
| 5 | 2.68 | 5.05 | 7.54 | 11.81 |
| 10 | 2.68 | 5.28 | 7.85 | 12.90 |
| 15 | 2.69 | 5.33 | 7.98 | 12.77 |

TABLE 10
R_{g} variation with CaCl₂

| | |
|---|---|
| Salt | |
| Concentration | |
| (weight %) | Radius of Gyration (nm) |

| CaCl₂ | 15K | 45K | 75K | 120K |
|---|---|---|---|---|
| 0 | 2.65 | 4.67 | 6.63 | 6.91 |
| 1 | 2.66 | 4.76 | 6.73 | 9.81 |
| 2.5 | 2.62 | 4.85 | 7.16 | 10.58 |
| 5 | 2.72 | 5.05 | 7.79 | 11.13 |
| 10 | 2.70 | 5.16 | 7.72 | 11.81 |
| 15 | 2.70 | 5.20 | 7.94 | 12.67 |

TABLE 11
R_{g} variation with Temperature

| | |
|---|---|
| Temperature | |
| (° C.) | Radius of Gyration (nm) |

| — | 15K | 45K | 75K | 120K |
|---|---|---|---|---|
| 25 | 2.65 | 4.67 | 6.63 | 6.91 |
| 40 | 2.64 | 4.75 | 6.69 | 7.76 |
| 60 | 2.67 | 4.75 | 6.86 | 8.64 |
| 80 | 2.70 | 4.80 | 6.90 | 10.13 |

TABLE 12
ν variation with NaCl

| | |
|---|---|
| Salt | |
| Concentration | |
| (weight %) | Excluded Volume Parameter |

| NaCl | 15K | 45K | 75K | 120K |
|---|---|---|---|---|
| 0 | 0.339 | 0.399 | 0.411 | 0.482 |
| 1 | 0.349 | 0.449 | 0.485 | 0.539 |
| 2.5 | 0.329 | 0.477 | 0.526 | 0.575 |
| 5 | 0.356 | 0.493 | 0.539 | 0.583 |
| 10 | 0.359 | 0.505 | 0.551 | 0.596 |
| 15 | 0.358 | 0.508 | 0.555 | 0.595 |

TABLE 13
ν variation with CaCl₂

| | |
|---|---|
| Salt | |
| Concentration | Excluded |
| (weight %) | Volume Parameter |

| CaCl₂ | 15K | 45K | 75K | 120K |
|---|---|---|---|---|
| 0 | 0.338 | 0.399 | 0.411 | 0.482 |
| 1 | 0.351 | 0.439 | 0.466 | 0.501 |
| 2.5 | 0.351 | 0.465 | 0.508 | 0.541 |
| 5 | 0.373 | 0.487 | 0.535 | 0.563 |
| 10 | 0.356 | 0.489 | 0.540 | 0.582 |
| 15 | 0.362 | 0.499 | 0.551 | 0.590 |

TABLE 14
ν variation with Temperature

| | |
|---|---|
| Temperature | Excluded |
| (° C.) | Volume Parameter |

| — | 15K | 45K | 75K | 120K |
|---|---|---|---|---|
| 25 | 0.339 | 0.399 | 0.411 | 0.482 |
| 40 | 0.332 | 0.417 | 0.434 | 0.472 |
| 60 | 0.342 | 0.424 | 0.453 | 0.491 |
| 80 | 0.351 | 0.437 | 0.467 | 0.498 |

These results are a clear manifestation of the antipolyelectrolyte phenomenon, but perhaps the most notable feature of FIG. 16 is the molecular weight dependency of the excluded volume parameter v. At 5% NaCl, for example, v was found to be 0.35 (globular) for 15K and 0.58 for 120K (swollen coil). These results are a departure from the scaling implied by equation 1, according to which v is a function only of solvent quality and not of molecular weight. This scaling however is only valid in the limit of large N, since it assumes a flexible chain with a large number of Kuhn segments. The Kuhn length calculated via the polymer excluded volume model of Benoit,

$$a = {( \frac{{R}_{g}^{2} ( 2 v + 1 ) ( 2 v + 2 )}{{N}^{2 v}} )}^{1 / 2} \qquad ( 6 )$$

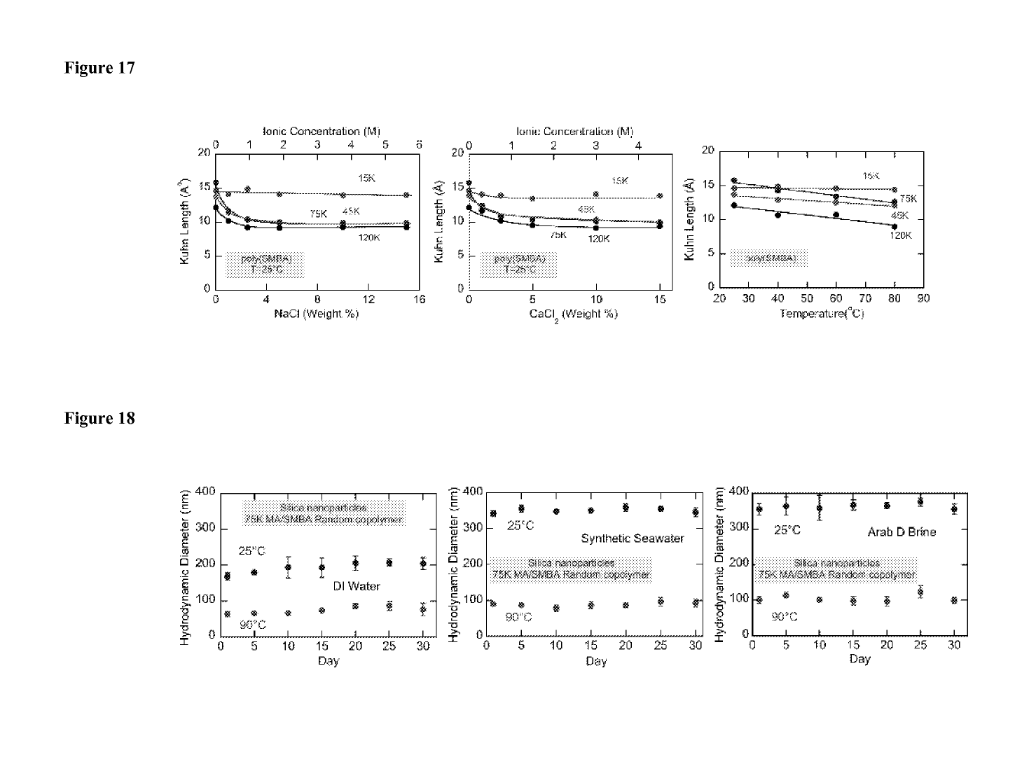

is shown in FIG. 17 as a function of salt, temperature and molecular weight. Poly(SBMA) was determined to have a Kuhn length between a˜15 Å and a˜9 Å (depending on salt concentrations, as discussed below), and thus the number of Kuhn segments lay between ˜2 (15K without added salt) to ˜14 (120K with 15 wt % CaCl₂). These values are clearly too low for the large N limit of equation 1 to be valid, and the decoupling between R_{g} and v observed in FIG. 16 is to be expected. An electrostatic contribution to a can also be anticipated since charge screening leads to a decrease in the length scale over which electrostatic interactions dominate (decrease in Debye length). This is a known phenomenon for polyelectrolytes, and the same effects were indeed observed for anti-polyelectrolyte based poly(SBMA).

It is interesting to contrast the behaviour of the lower molecular weight 15K with that of the higher molecular weight polymers 45K to 120K observed in FIGS. 16 and 17. The 15K homopolymer has a constant Kuhn length and an invariant globular shape, while the 45K-120K systems show a steady decrease in a and a clear transition towards more swollen conformations. Such a molecular weight dependence of a and v is surprising, but can be rationalized on the basis of the average distance l between an interacting pair of ammonium and sulfonate groups confined within a polyzwitterion coil. This average spacing is known to increase with molecular weight. Therefore for a densely packed globular chain such as 15K, l is very small, and attractive ionic interactions dominate over charge screening, leading to retention of the strong globular structure and constant Kuhn length. For 45K-120K systems on the other hand, monomeric units are less densely packed, and thus allow charge screening to dominate over electrostatic interactions. Since chain swelling is a result of reduced ionic correlations, electrolyte addition leads to a more flexible coil and a decrease in a.

Another notable feature is the relative effect of NaCl vs. CaCl₂. While previous studies have established an order of solubilization for different salt cation and anion structures, a preferential order for R_{g} and v sensitivity has not been established. FIG. 16 implies that the divalent Ca²⁺ is a much stronger cation than is monovalent Na⁻ when compared on a cumulative ionic strength basis rather than on a mass percent basis.

### Colloidal Stability

The SANS results indicate that SBMA has an osmotic response opposite that of polyelectrolytes. This is unique in the context of colloidal applications, since it enables the design of a steric layer that strengthens rather than weakens suspension stability in response to increasingly harsh reservoir conditions (FIG. 14). Additionally, SBMA is a good candidate due to its hydrophilic backbone (amide group) and hydrolytic stability (methacrylamide>acrylamide>methacrylate>acrylate). Silica was initially chosen as the model core because of its chemical and thermal resistance to reservoir conditions. Immobilization of the polymeric layer prevents diffusive desorption and thermal degradation of the attached polymers, and was easily accomplished via carbodiimide conjugation to the particle surface. This conjugation was facilitated by incorporation of acid sites on the polymeric backbone, which was achieved by copolymerization of MA with SBMA. Since the distribution of acid sites along the polymer backbone can be expected to affect grafting density, geometry and cumulative repulsion offered by the steric layer significantly, two polymeric architectures were designed: a free radical copolymer with randomly distributed acid sites, and a RAFT-based block copolymer with a segregated acid block.

### Random Copolymer

The random copolymer, a hybrid of a polyelectrolyte (MA) and an antipolyelectrolyte (SBMA), is easily synthesized. FIG. 18 shows the stability of random copolymer-functionalized silica nanoparticles in DI water, synthetic seawater and Arab-D brine at two different heat-ageing temperatures. Temperature dependent clustering was observed, with a smaller particle size at 90° C. and a larger particle size at 25° C. The clustering at lower temperatures was likely due to an insufficient hydration of the SBMA units to counter the relatively poor solvation of MA in an electrolyte rich environment. At elevated temperatures on the other hand, increased SBMA solvation provided the necessary steric repulsion and led to deaggregation of the clusters.

### Block Copolymer

In order to overcome the temperature dependent clustering observed with random copolymer stabilizers, a block copolymer with a segregated MA anchor block was designed. The segregation of the anchor block is useful for at least two reasons: it allows for a higher graft density, and for exclusion of MA from the steric layer. High graft densities are known to introduce repulsive entropic effects via stretched chain compression, while exclusion of MA from the steric layer was expected to overcome the issue of competing osmotic effects. Overlayed chromatograms of the block copolymers prepared at four different molecular weight of the SBMA block are shown in FIG. 22, and polydispersities are reported in Table 15.

TABLE 15
MA/SBMA Block Copolymer Molecular Weight and
Polydispersity Synthesized via RAFT Polymerization

| Sample | Mₙ (g/mol) | Polydispersity |
|---|---|---|
| 15K-Block | 13,750 | 1.227 |
| 35K-Block | 36,920 | 1.035 |
| 75K-Block | 74,990 | 1.092 |
| 120K-Block | 124,700 | 1.023 |

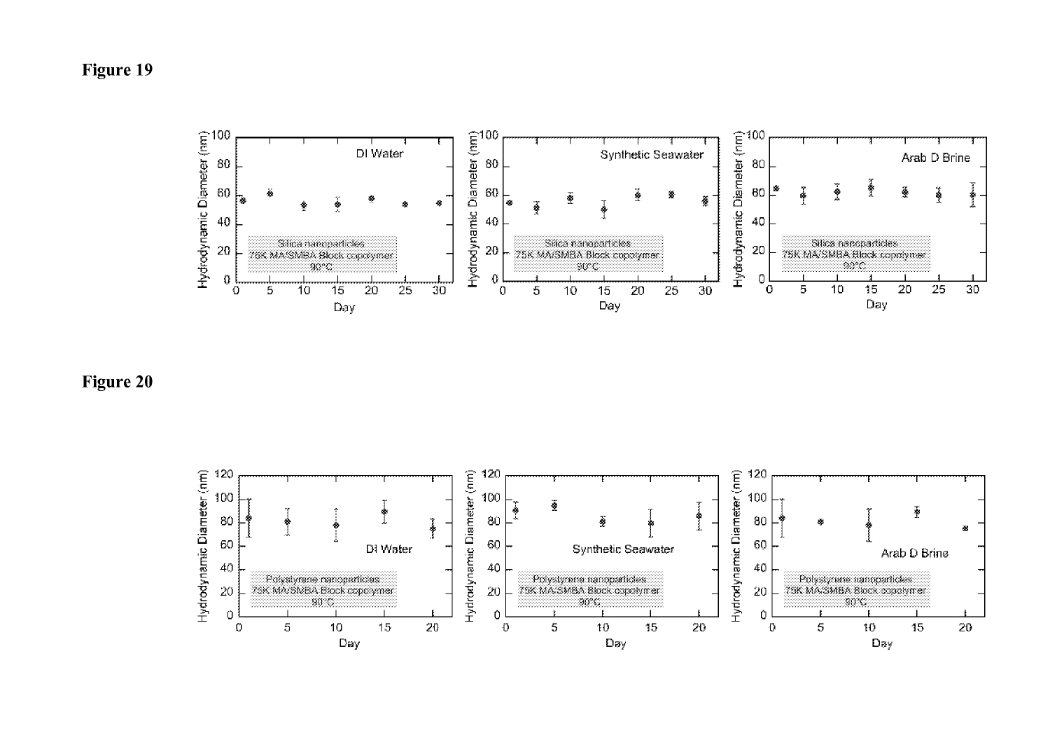

SANS studies indicated that larger SBMA blocks (75K-120K) expand in size (R_{g}) and swell (v) more compared to smaller and more globular SBMA blocks (15K-45K). Thus, 75K-120K blocks were expected to perform better as stabilizing agents. Therefore the stability of particles functionalized with the 75K-Block polymer was investigated in DI water, synthetic seawater, and Arab-D Brine; the results are given in FIG. 19. No temperature dependent clustering was observed, and uniform long-term stability was achieved over the entire desired temperature and salt ranges.

The results shown in FIG. 19 were obtained with the nanoparticles dispersed in polypropylene vials. When glass vials were used to contain the suspensions, the silica nanoparticles were found to be colloidally unstable following exposure to elevated temperatures for a week. The instability was likely due to strong interactions between the silica core and the glass vial surface, thereby providing the nanoparticles with a favourable site for adsorption and eventual aggregation. That this behaviour was not observed in polypropylene vials was attributed to the surface hydrophobicity and weak interaction between the vial surface and silica core. To test this hypothesis, a hydrophobic, amine-functionalized polystyrene core (28 nm) was functionalized with the 75K-Block copolymer via carbodiimide conjugation. With these particles, long-term colloidal stability was observed in both glass and polypropylene vials, indicating that the steric barrier was sufficient to overcome any interaction with glass or polypropylene surfaces. Long-term stability results are shown in FIG. 20.

The results presented above are of significant importance on three accounts. Firstly, they demonstrate a novel utilization of the antipolyelectrolyte phenomenon for developing colloidal stabilizers that actually strengthen in their performance with increasing harshness of reservoir conditions. Secondly, there are a large number of synthetic polybetaines that could be engineered similarly to serve as polymeric stabilizers, thereby extending the design space for utilizing the antipolyelectrolyte effect in the stabilization of nanoparticle suspensions. Thirdly, it significantly extends the range of electrolyte concentrations over which long-term colloidal stability can be achieved for reservoir applications. No other studies have reported successful long-term stabilization in Arab-D or equivalently harsh brine. Furthermore, the coating shows excellent stability at significantly higher concentrations of Mg²⁺ and Ca²⁺ than has been previously reported.

### Summary

The key deficiency of polyelectrolyte-based colloidal stabilization in strong brines (double layer collapse) has been overcome by designing anti-polyelectrolyte based stabilizers that advantageously utilize the enhanced charge screening afforded by harsh reservoir conditions. The effect was quantified using SANS for a series of low polydispersity homopolymers of poly(sulfobetaine methacrylamide) prepared via RAFT polymerization, and significant molecular weight dependencies on size and shape were observed. The larger sized polymers (75K-120K) were noted to be optimal for stabilizing nanoparticles under harsh reservoir conditions, and successful stabilization was achieved under significantly harsher conditions (including at high concentrations of divalent ions) than previously reported. Additionally, the approach proved to be versatile in terms of the being applicable to hydrophilic as well as hydrophobic nanoparticles.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A composite nanoparticle, comprising a core; and an ionic polymer.

2. The composite nanoparticle of claim 1, wherein the ionic polymer comprises a plurality of repeat units having Formula II: 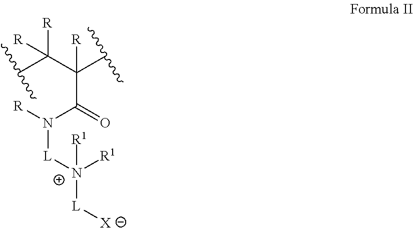
or a salt thereof,
wherein, independently for each occurrence,
R is hydrogen or branched or unbranched, substituted or unsubstituted alkyl;
R¹ is hydrogen or branched or unbranched, substituted or unsubstituted alkyl;
L is branched or unbranched, substituted or unsubstituted alkylene, or substituted or unsubstituted arylene; and
X is —CO₂, —SO₃, —PO₃H, or —PO₂R.

3. The composite nanoparticle of claim 2, wherein the ionic polymer further comprises a plurality of repeat units having Formula IIIa or Formula IVa, and a plurality of repeat units having Formula IIIb or Formula IVb: 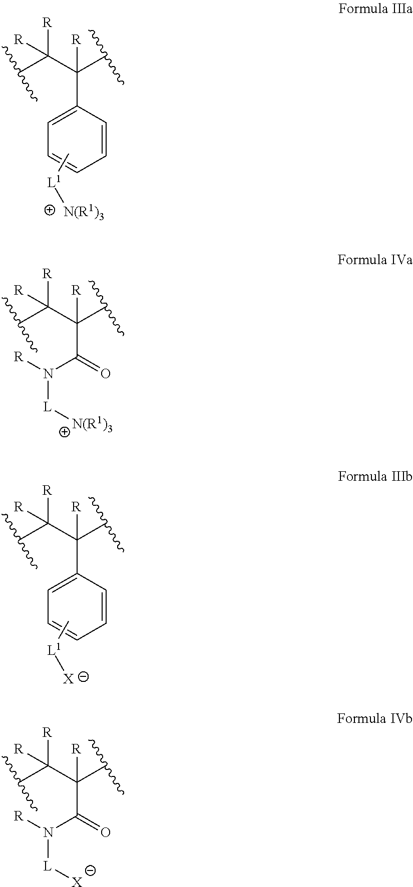
or a salt of any of them,
wherein, independently for each occurrence,
L¹ is absent, or a branched or unbranched, substituted or unsubstituted alkylene.

4-7. (canceled)

8. The composite nanoparticle of claim 1, wherein the ionic polymer comprises a plurality of repeat units having Formula IIIa or Formula IVa, and a plurality of repeat units having Formula IIIb or Formula IVb: 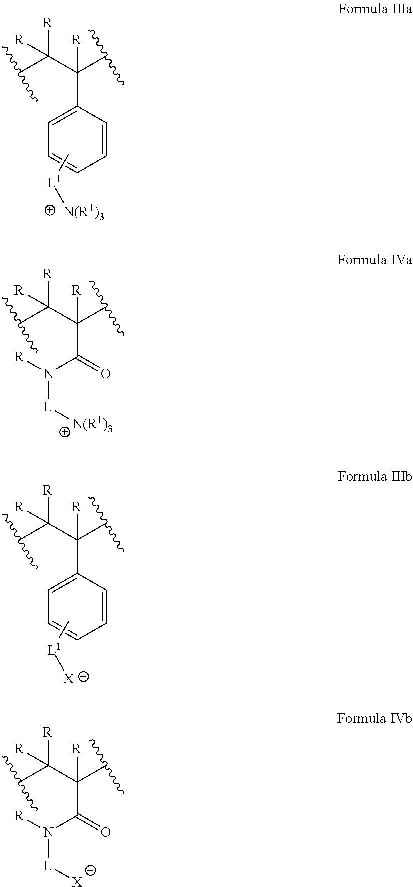
or a salt of any of them,
wherein, independently for each occurrence,
R is hydrogen or branched or unbranched, substituted or unsubstituted alkyl;
R¹ is hydrogen or branched or unbranched, substituted or unsubstituted alkyl;
L is branched or unbranched, substituted or unsubstituted alkylene, or substituted or unsubstituted arylene;
L¹ is absent, or a branched or unbranched, substituted or unsubstituted alkylene; and
X is —CO₂, —SO₃, —PO₃H, or —PO₂R.

9-12. (canceled)

13. The composite nanoparticle of claim 2, wherein the ionic polymer further comprises a plurality of repeat units having Formula I: 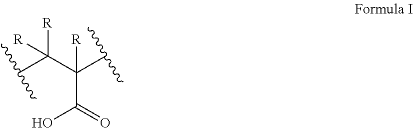
or a salt thereof,
wherein, independently for each occurrence,
R is hydrogen or branched or unbranched, substituted or unsubstituted alkyl.

14-29. (canceled)

30. The composite nanoparticle of claim 1, wherein the ionic polymer is covalently bonded to the core.

31. The composite nanoparticle of claim 1, wherein the ionic polymer is covalently bonded to the core via a linker.

32-36. (canceled)

37. The composite nanoparticle of claim 1, wherein the core is solid.

38. The composite nanoparticle of claim 1, wherein the core is selected from the group consisting of silica, carbon black, carbon nanotubes, graphene, iron, and magnetite.

39. (canceled)

40. The composite nanoparticle of claim 1, further comprising a reporter molecule.

41. (canceled)

42. An aqueous mixture, comprising a plurality of composite nanoparticles; wherein the composite nanoparticles comprise a core, and an ionic polymer.

43-45. (canceled)

46. The aqueous mixture of claim 42, wherein the composite nanoparticles form a suspension in the aqueous mixture.

47. (canceled)

48. A method of detecting the presence of, the concentration of, or the location of an analyte in a substrate, comprising the steps of:
contacting the substrate and a composition, wherein the composition comprises a plurality of reporter molecules at a first concentration, and a plurality of composite nanoparticles; wherein the composite nanoparticles comprise a core, and an ionic polymer;
after a period of time, recovering a subset of the composite nanoparticles; and
determining the concentration of the reporter molecules in the subset of composite nanoparticles.

49. The method of claim 48, wherein at least a portion of the reporter molecules is releasable from the composite nanoparticles upon exposure to the analyte.

50. The method of claim 48, wherein the reporter molecule is non-covalently associated with the composite nanoparticle.

51. The method of claim 48, wherein the substrate is an oilfield or an oil well.

52. The method of claim 48, wherein the analyte is an organic compound, an inorganic compound, an ion, or a heavy metal.

53. The method of claim 48, wherein the analyte is petroleum.

54. The method of claim 48, wherein the analyte is hydrogen sulfide.

55. The composite nanoparticle of claim 8, wherein the ionic polymer further comprises a plurality of repeat units having Formula I: 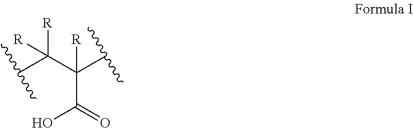
or a salt thereof,
wherein, independently for each occurrence,
R is hydrogen or branched or unbranched, substituted or unsubstituted alkyl.
